SEQUENCE LISTING (25 sequences)

SEQ ID NO: 1 - DNA, 40 bp, Homo sapiens
tgccaggctctggagggtcggaggaagctggcccagttgc

SEQ ID NO: 2 - DNA, 40 bp, Homo sapiens
acctgaagagatgccagcaggaggaagctggcccagttgc

SEQ ID NO: 3 - DNA, 40 bp, Homo sapiens
ggagaatcaagagctccgagggaatgcagctggaagatct

SEQ ID NO: 4 - DNA, 1104 bp, Homo sapiens
atgaataggcacctctggaagagccaactgtgtgagatggtgcagcccagtggtggcccggcagcagatcaggacgtactgggcgaagagtctcctctggggaagccagccatgctgcacctgccttcagaacagggcgctcctgagaccctccagcgctgcctggaggagaatcaagagctccgagatgccatccggcagagcaaccagattctgcgggagcgctgcgaggagcttctgcatttccaagccagccagagggaggagaaggagttcctcatgtgcaagttccaggaggccaggaaactggtggagagactcggcctggagaagctcgatctgaagaggcagaaggagcaggctctgcgggaggtggagcacctgaagagatgccagcagcagatggctgaggacaaggcctctgtgaaagcccaggtgacgtccttgctcggggagctgcaggagagccagagtcgcttggaggctgccactaaggaatgccaggctctggagggtcggaggaagctggcccagttgcaggtggcctatcaccagctcttccaagaatacgacaaccacatcaagagcagcgtggtgggcagtgagcggaagcgaggaatgcagctggaagatctcaaacagcagctccagcaggccgaggaggccctggtggccaaacaggaggtgatcgataagctgaaggaggaggccgagcagcacaagattgtgatggagaccgttccggtgctgaaggcccaggcggatatctacaaggcggacttccaggctgagaggcaggcccgggagaagctggccgagaagaaggagctcctgcaggagcagctggagcagctgcagagggagtacagcaaactgaaggccagctgtcaggagtcggccaggatcgaggacatgaggaagcggcatgtcgaggtctcccaggcccccttgccccccgcccctgcctacctctcctctcccctggccctgcccagccagaggaggagcccccccgaggagccacctgacttctgctgtcccaagtgccagtatcaggcccctgatatggacaccctgcagatacatgtcatggagtgcattgag

SEQ ID NO: 5 - protein, 368 aa, Homo sapiens
MNRHLWKSQLCEMVQPSGGPAADQDVLGEESPLGKPAMLHLPSEQGAPETLQRCLEENQELRDAIRQSNQILRERCEELLHFQASQREEKEFLMCKFQEARKLVERLGLEKLDLKRQKEQALREVEHLKRCQQQMAEDKASVKAQVTSLLGELQESQSRLEAATKECQALEGRRKLAQLQVAYHQLFQEYDNHIKSSVVGSERKRGMQLEDLKQQLQQAEEALVAKQEVIDKLKEEAEQHKIVMETVPVLKAQADIYKADFQAERQAREKLAEKKELLQEQLEQLQREYSKLKASCQESARIEDMRKRHVEVSQAPLPPAPAYLSSPLALPSQRRSPPEEPPDFCCPKCQYQAPDMDTLQIHVMECIE

SEQ ID NO: 6 - DNA, 483 bp, Homo sapiens
atgaataggcacctctggaagagccaactgtgtgagatggtgcagcccagtggtggcccggcagcagatcaggacgtactgggcgaagagtctcctctggggaagccagccatgctgcacctgccttcagaacagggcgctcctgagaccctccagcgctgcctggaggagaatcaagagctccgagatgccatccggcagagcaaccagattctgcgggagcgctgcgaggagcttctgcatttccaagccagccagagggaggagaaggagttcctcatgtgcaagttccaggaggccaggaaactggtggagagactcggcctggagaagctcgatctgaagaggcagaaggagcaggctctgcgggaggtggagcacctgaagagatgccagcaggaggaagctggcccagttgcaggtggcctatcaccagctcttccaagaatacgacaaccacatcaagagcagcgtggtgggcag

SEQ ID NO: 7 - protein, 161 aa, Homo sapiens
MNRHLWKSQLCEMVQPSGGPAADQDVLGEESPLGKPAMLHLPSEQGAPETLQRCLEENQELRDAIRQSNQILRERCEELLHFQASQREEKEFLMCKFQEARKLVERLGLEKLDLKRQKEQALREVEHLKRCQQEEAGPVAGGLSPALPRIRQPHQEQRGGQ

SEQ ID NO: 8 - DNA, 486 bp, Homo sapiens
atgcagctggaagatctcaaacagcagctccagcaggccgaggaggccctggtggccaaacaggaggtgatcgataagctgaaggaggaggccgagcagcacaagattgtgatggagaccgttccggtgctgaaggcccaggcggatatctacaaggcggacttccaggctgagaggcaggcccgggagaagctggccgagaagaaggagctcctgcaggagcagctggagcagctgcagagggagtacagcaaactgaaggccagctgtcaggagtcggccaggatcgaggacatgaggaagcggcatgtcgaggtctcccaggcccccttgccccccgcccctgcctacctctcctctcccctggccctgcccagccagaggaggagcccccccgaggagccacctgacttctgctgtcccaagtgccagtatcaggcccctgatatggacaccctgcagatacatgtcatggagtgcattgag

SEQ ID NO: 9 - protein, 162 aa, Homo sapiens [sequence omitted]

SEQ ID NO: 10 - DNA, 264 bp, Homo sapiens
atgaataggcacctctggaagagccaactgtgtgagatggtgcagcccagtggtggcccggcagcagatcaggacgtactgggcgaagagtctcctctggggaagccagccatgctgcacctgccttcagaacagggcgctcctgagaccctccagcgctgcctggaggagaatcaagagctccgagggaatgcagctggaagatctcaaacagcagctccagcaggccgaggaggccctggtggccaaacaggaggtgatcga

SEQ ID NO: 11 - protein, 88 aa, Homo sapiens
MNRHLWKSQLCEMVQPSGGPAADQDVLGEESPLGKPAMLHLPSEQGAPETLQRCLEENQELRGNAAGRSQTAAPAGRGGPGGQTGGDR

SEQ ID NO: 12 - DNA, 20 bp, Homo sapiens
tggagggtcggaggaagctg

SEQ ID NO: 13 - DNA, 20 bp, Homo sapiens
atgccagcaggaggaagctg

SEQ ID NO: 14 - DNA, 20 bp, Homo sapiens
gagctccgagggaatgcagc

SEQ ID NO: 15 - protein, 10 aa, Homo sapiens
QALEGRRKLA

SEQ ID NO: 16 - protein, 10 aa, Homo sapiens
KRCQQEEAGP

SEQ ID NO: 17 - protein, 10 aa, Homo sapiens
MQLEDLKQQL

SEQ ID NO: 18 - protein, 10 aa, Homo sapiens
NQELRGNAAG

SEQ ID NO: 19 - DNA, 28 bp, Homo sapiens
tgttggatgaataggcacctctggaaga

SEQ ID NO: 20 - DNA, 28 bp, Homo sapiens
ttcagcttatcgatcacctcctgtttgg

SEQ ID NO: 21 - DNA, 28 bp, Homo sapiens
atgaataggcacctctggaagagccaac

SEQ ID NO: 22 - DNA, 27 bp, Homo sapiens
atgcagctggaagatctcaaacagcag

SEQ ID NO: 23 - DNA, 27 bp, Homo sapiens
ctactcaatgcactccatgacatgtat

SEQ ID NO: 24 - DNA, 27 bp, Homo sapiens
tcactgcccaccacgctgctcttgatg

SEQ ID NO: 25 - DNA, 27 bp, Homo sapiens
ttatcgatcacctcctgtttggccacc

(19) United States
(12) Patent Application Publication
(10) Pub. No.: US 2004/0175797 A1
(43) Pub. Date: Sep. 9, 2004

(54) Alternatively spliced isoforms of inhibitor of kappa-B kinase gamma (IKBKG)

(76) Inventors: Jason M. Johnson, Seattle, WA (US); Philip W. Garrett-Engele, Seattle, WA (US); Zhengyan Kan, Bellevue, WA (US)

Correspondence Address: R. Douglas Bradley, Merck & Co., Inc., P.O. Box 2000, Patent Department RY60-30, Rahway, NJ, 07065-0907, US

(21) Appl. No.: 10/792,063

(22) Filed: Mar. 3, 2004

Related U.S. Application Data
(60) Provisional application No. 60/452,293, filed on Mar. 4, 2003.

(51) Int. Cl. C12N 9/12; C07H 21/04

(52) U.S. Cl. 435/69.1; 435/194; 435/320.1; 435/325; 536/23.2

(57) ABSTRACT

The present invention features nucleic acids and polypeptides encoding four novel splice variant isoforms of inhibitor of kappa light polypeptide gene enhancer in B cells, kinase of, gamma (IKBKG). The polynucleotide sequences of IKBKGsv1, IKBKGsv2.1, IKBKGsv2.2, and IKBKGsv3 are provided by SEQ ID NO 4, SEQ ID NO 6, SEQ ID NO 8, and SEQ ID NO 10, respectively. The amino acid sequences for IKBKGsv1, IKBKGsv2.1, IKBKGsv2.2, and IKBKGsv3 are provided by SEQ ID NO 5, SEQ ID NO 7, SEQ ID NO 9, and SEQ ID NO 11, respectively. The present invention also provides methods for using IKBKGsv1, IKBKGsv2.1, IKBKGsv2.2, and IKBKGsv3 polynucleotides and proteins to screen for compounds that bind to IKBKGsv1, IKBKGsv2.1, IKBKGsv2.2, and IKBKGsv3, respectively.

This application claims priority to U.S. Provisional Patent Application Serial No. 06/452,293 filed on Mar. 4, 2003, which is incorporated by reference herein in its entirety.

### BACKGROUND OF THE INVENTION

The references cited herein are not admitted to be prior art to the claimed invention.

The transcription factor nuclear factor kappa beta (NF-kappa-B) plays an integral role in the cellular response to a wide array of harmful stimuli, including cytokines, such as tumor necrosis factor alpha (TNF-1) and interlukin-1 (IL-1), bacterial LPS, viral infection, phorbol esters, UV radiation, and free radicals. NF-kappa-B regulates genes involved in immune function, inflammation responses, growth control, cell death, cell adhesion, and viral replication (for reviews see Baldwin, A. S., 1996, Annu. Rev. Immunol. 14, 649-681; Baeuerle, P. A. & Baltimore, D., 1996, Cell 87, 13-20; Stancovski, I. & Baltimore, D., 1997 Cell, 91, 299-302). The function of NF-kappa-B has been implicated in diseases as varied as rheumatoid arthritis, lupus, HIV-AIDS, influenza, septic shock, atherosclerosis, oncogenesis, and apoptosis (Baldwin, 1996).

In its inactive state, NF-kappa-B resides in the cytoplasm, bound to an inhibitory protein, 1-kappa-B. In response to stimuli, 1-kappa-B is phosphorylated, marking it for ubiquitination and proteosome-mediated degradation. The degradation of 1-kappa leads to release of NF-kappa-B which is then translocated to the nucleus where it activates transcription of target genes (Stancovski, 1997). A 500-900 kD kinase complex is responsible for the phosphorylation of 1-kappa-B (Chen, et. al., 1995, Genes Dev. 9, 1586-1597; DiDonata, et. al., 1997, Nature 388, 548-554; Zandi, et. al., 1997, Cell 91, 243-252). The kinase complex, 1-kappa-B-kinase (IKK) is composed of two catalytic subunits, IKK-alpha and IKK-beta, both of which are essential for the phosphorylation of 1-kappa-B and the activation of NF-kappa-B (Zandi, 1997).

A third subunit of the IKK complex, having a regulatory role, has been identified by a number of groups (Rothwarf, et. al., 1998, Nature 395, 297-300; Mercurio, et. al., 1999, Mol. and Cell. Biol. 19, 1526-1538; Yongan, et. al., 1999, Immunology 96, 1042-1047). This third subunit is alternately known as I-kappa-B-kinase-gamma (IKBKG or IKK-gamma), IKK associated protein (IKKAP-1), FIP-3, or NF-kappa-B essential modulator (NEMO). The structural motifs of IKBKG include two coiled-coil motifs, a leucine zipper, and a putative zinc finger (Makris, et. al., 2002, Mol. and Cell. Biol. 22, 6573-6581). IKBKG lacks catalytic activity, but is essential for the activation of the IKK complex and NF-kappa-B function (Yamaoka, et. al., 1998, Cell 93, 1231-1240). The amino terminus of IKBKG is essential for assembly of the kinase complex and the carboxy terminal region is required for activation of the IKK complex (Makris, et. al., 2002, Mol. and Cell. Biol. 22, 6573-6581).

Several inhibitors of NF-kappa-B have been identified and shown to be effective therapeutics. These include tepoxalin (a dual inhibitor of cyclooxygenase and 5-lipoxygenase), interlukin-10, glucocorticoids (immunosuppressants such as prednisone and dexamethasone which act to upregulate levels of 1-kappa-B-alpha thereby decreasing the amount of NF-kappa-B that can translocate to the nucleus), salicylates, cyclosporin and rapamycin, and nitric oxide (also upregulates I-kappa-B-alpha) (Baldwin, A. S., 1996, Annu. Rev. Immunol. 14, 649-681).

IKK activation appears to be regulated by phosphorylation, in that IKK activity is inhibited by protein phosphatase 2A (PP2A) (DiDonato, et. al., 1997, Nature 388, 548-554). IKBKG is phosphorylated in response to cytokine stimulants and it has been suggested that PP2A could be used to inhibit phosphorylation of IKBKG and activation of the IKK complex (Prajapati, S. and Gaynor, R., 2002, J. Bio. Chem. 277, 24331-24339). Recently, a highly selective inhibitor of 1-kappa-B-kinase catalytic subunits, designated BMS-345541 (4(2′-aminoethyl)amino-1,8-dimethylimidazo(1,2-a)quinoxaline), was identified as an inhibitor of 1-kappa-B phosphorylation and NF-kappa-B activated transcription of inflammatory cytokines in mice (Burke, et. al., 2003, J. Biol. Chem. 278, 1450-1456). In addition, antisense IKBKG oligonucleotides have been shown to inhibit NF-kappa-B activation (Rothwarf, et. al., 1998, Nature 395, 297-300; Krappmann, et. al., 2000, J. Biol. Chem. 38, 29779-29787).

The specific region within IKK-beta required for the association of IKBKG with the IKK complex was recently identified as a six amino acid carboxy-terminal region and termed the NEMO-binding domain (NBD) (May, et. al., 2000, Science 289, 1550-1554). A cell permeable peptide spanning the IKK-beta NBD inhibits the association of IKK-beta with IKBKG (NEMO) and translocation of NF-kappa-B to the nucleus in response to TNF-alpha stimulation in vitro, while also increasing basal NF-kappa-B activity. In experimental mouse model systems of acute inflammation, administration of NBD peptides resulted in an anti-inflammatory response that was as effective as treatment with dexamethasone. (May, et. al., 2000). These results indicate the potential efficacy of drugs targeting IKBKG. It has been suggested that such drugs may be of clinical importance in that they act by preventing association of the IKK complex while maintaining basal NF-kappa-B activity, thereby potentially avoiding toxic side effects. (May, et. al., 2000).

Because of the multiple therapeutic values of drugs targeting the NF-kappa-B pathway, and the essential regulatory role played by IKBKG, there is a need in the art for compounds that selectively bind to isoforms of IKBKG. The present invention is directed towards three novel IKBKG isoforms (IKBKGsv1, IKBKGsv2, and IKBKGsv3) and uses thereof.

### BRIEF DESCRIPTION OF THE FIGURES

FIG. 1A illustrates the exon structure of IKBKG mRNA corresponding to the known long reference form of IKBKG mRNA (labeled NM_{—}003639) and the exon structure corresponding to the inventive short form splice variants (labeled IKBKGsv1, IKBKGsv2, and IKBKGsv3). FIG. 1B depicts the nucleotide sequences of the exon junctions resulting from the splicing of exon 4 to exon 6 in the case of IKBKGsv1 mRNA [SEQ ID NO 1]; the splicing of exon 3 to exon 6 in the case of the IKBKGsv2 mRNA [SEQ ID NO 2]; and the splicing of exon 2 to exon 7 in the case of IKBKGsv3 mRNA [SEQ ID NO 3]. In FIG. 1B, in the case of the IKBKGsv1 splice junction sequence [SEQ ID NO 1], the nucleotides shown in italics represent the 20 nucleotides at the 3′ end of exon 4 and the nucleotides shown in underline represent the 20 nucleotides at the 5′ end of exon 6; in the case of the IKBKGsv2 splice junction sequence [SEQ ID NO 2], the nucleotides shown in italics represent the 20 nucleotides at the 3′ end of exon 3 and the nucleotides shown in underline represent the 20 nucleotides at the 5′ end of exon 6; and in the case of the IKBKGsv3 splice junction sequence [SEQ ID NO 3], the nucleotides shown in italics represent the 20 nucleotides at the 3′ end of exon 2 and the nucleotides shown in underline represent the 20 nucleotides at the 5′ end of exon 7.

### SUMMARY OF THE INVENTION

Microarray experiments and RT-PCR have been used to identify and confirm the presence of novel splice variants of human IKBKG mRNA. More specifically, the present invention features polynucleotides encoding different protein isoforms of IKBKG. A polynucleotide sequence encoding IKBKGsv1 is provided by SEQ ID NO 4. An amino acid sequence for IKBKGsv1 is provided by SEQ ID NO 5. A polynucleotide sequence encoding IKBKGsv2.1 is provided by SEQ ID NO 6. An amino acid sequence for IKBKGsv2.1 is provided by SEQ ID NO 7. A polynucleotide sequence encoding IKBKGsv2.2 is provided by SEQ ID NO 8. An amino acid sequence for IKBKGsv2.2 is provided by SEQ ID NO 9. A polynucleotide sequence encoding IKBKGsv3 is provided by SEQ ID NO 10. An amino acid sequence for IKBKGsv3 is provided by SEQ ID NO 11.

Thus, a first aspect of the present invention describes a purified IKBKGsv1 encoding nucleic acid, a purified IKBKGsv2.1 encoding nucleic acid, a purified IKBKGsv2.2 encoding nucleic acid, and a purified IKBKGsv3 encoding nucleic acid. The IKBKGsv1 encoding nucleic acid comprises SEQ ID NO 4 or the complement thereof. The IKBKGsv2.1 encoding nucleic acid comprises SEQ ID NO 6 or the complement thereof. The IKBKGsv2.2 encoding nucleic acid comprises SEQ ID NO 8 or the complement thereof. The IKBKGsv3 encoding nucleic acid comprises SEQ ID NO 10 or the complement thereof. Reference to the presence of one region does not indicate that another region is not present. For example, in different embodiments the inventive nucleic acid can comprise, consist, or consist essentially of an encoding nucleic acid sequence of SEQ ID NO 4, can comprise, consist, or consist essentially of the nucleic acid sequence of SEQ ID NO 6, can comprise, consist, or consist essentially of the nucleic acid sequence of SEQ ID NO 8, or alternatively can comprise, consist, or consist essentially of the nucleic acid sequence of SEQ ID NO 10.

Another aspect of the present invention describes a purified IKBKGsv1 polypeptide that can comprise, consist or consist essentially of the amino acid sequence of SEQ ID NO 5. An additional aspect describes a purified IKBKGsv2.1 polypeptide that can comprise, consist, or consist essentially of the amino acid sequence of SEQ ID NO 7. An additional aspect describes a purified IKBKGsv2.2 polypeptide that can comprise, consist, or consist essentially of the amino acid sequence of SEQ ID NO 9. An additional aspect describes a purified IKBKGsv3 polypeptide that can comprise, consist, or consist essentially of the amino acid sequence of SEQ ID NO 11.

Another aspect of the present invention describes expression vectors. In one embodiment of the invention, the inventive expression vector comprises a nucleotide sequence encoding a polypeptide comprising, consisting, or consisting essentially of SEQ ID NO 5, wherein the nucleotide sequence is transcriptionally coupled to an exogenous promoter. In another embodiment, the inventive expression vector comprises a nucleotide encoding a polypeptide comprising, consisting, or consisting essentially of SEQ ID NO 7, wherein the nucleotide sequence is transcriptionally coupled to an exogenous promoter. In another embodiment, the inventive expression vector comprises a nucleotide encoding a polypeptide comprising, consisting, or consisting essentially of SEQ ID NO 9, wherein the nucleotide sequence is transcriptionally coupled to an exogenous promoter. In another embodiment, the inventive expression vector comprises a nucleotide encoding a polypeptide comprising, consisting, or consisting essentially of SEQ ID NO 11, wherein the nucleotide sequence is transcriptionally coupled to an exogenous promoter.

Alternatively, the nucleotide sequence comprises, consists, or consists essentially of SEQ ID NO 4, and is transcriptionally coupled to an exogenous promoter. In another embodiment, the nucleotide sequence comprises, consists, or consists essentially of SEQ ID NO 6, and is transcriptionally coupled to an exogenous promoter. In another embodiment, the nucleotide sequence comprises, consists, or consists essentially of SEQ ID NO 8, and is transcriptionally coupled to an exogenous promoter. In another embodiment, the nucleotide sequence comprises, consists, or consists essentially of SEQ ID NO 10, and is transcriptionally coupled to an exogenous promoter.

Another aspect of the present invention describes recombinant cells comprising expression vectors comprising, consisting, or consisting essentially of the above-described sequences and the promoter is recognized by an RNA polymerase present in the cell. Another aspect of the present invention, describes a recombinant cell made by a process comprising the step of introducing into the cell an expression vector comprising a nucleotide sequence comprising, consisting, or consisting essentially of SEQ ID NO 4, SEQ ID NO 6, SEQ ID NO 8, or SEQ ID NO 10, or a nucleotide sequence encoding a polypeptide comprising, consisting, or consisting essentially of an amino acid sequence of SEQ ID NO 5, SEQ ID NO 7, SEQ ID NO 9, or SEQ ID NO 11, wherein the nucleotide sequence is transcriptionally coupled to an exogenous promoter. The expression vector can be used to insert recombinant nucleic acid into the host genome or can exist as an autonomous piece of nucleic acid.

Another aspect of the present invention describes a method of producing IKBKGsv1, IKBKGsv2.1, IKBKGsv2.2, or IKBKGsv3 polypeptide comprising SEQ ID NO 5, SEQ ID NO 7, SEQ ID NO 9, or SEQ ID NO 11, respectively. The method involves the step of growing a recombinant cell containing an inventive expression vector under conditions wherein the polypeptide is expressed from the expression vector.

Another aspect of the present invention features a purified antibody preparation comprising an antibody that binds selectively to IKBKGsv1 as compared to one or more IKBKG isoform polypeptides that are not IKBKGsv1. In another embodiment, a purified antibody preparation is provided comprising antibody that binds selectively to IKBKGsv2.1 as compared to one or more IKBKG isoform polypeptides that are not IKBKGsv2.1. In another embodiment, a purified antibody preparation is provided comprising antibody that binds selectively to IKBKGsv2.2 as compared to one or more IKBKG isoform polypeptides that are not IKBKGsv2.2. In another embodiment, a purified antibody preparation is provided comprising antibody that binds selectively to IKBKGsv3 as compared to one or more IKBKG isoform polypeptides that are not IKBKGsv3.

Another aspect of the present invention provides a method of screening for a compound that binds to IKBKGsv1, IKBKGsv2.1, IKBKGsv2.2, IKBKGsv3 or fragments thereof. In one embodiment, the method comprises the steps of: (a) expressing a polypeptide comprising the amino acid sequence of SEQ ID NO 5 or a fragment thereof from recombinant nucleic acid; (b) providing to said polypeptide a labeled IKBKG ligand that binds to said polypeptide and a test preparation comprising one or more test compounds; (c) and measuring the effect of said test preparation on binding of said test preparation to said polypeptide comprising SEQ ID NO 5. Alternatively, this method could be performed using SEQ ID NO 7, SEQ ID NO 9, or SEQ ID NO 11, instead of SEQ ID NO 5.

In another embodiment of the method, a compound is identified that binds selectively to IKBKGsv1 polypeptide as compared to one or more IKBKG isoform polypeptides that are not IKBKGsv1. This method comprises the steps of: providing a IKBKGsv1 polypeptide comprising SEQ ID NO 5; providing a IKBKG isoform polypeptide that is not IKBKGsv1, contacting said IKBKGsv1 polypeptide and said IKBKG isoform polypeptide that is not IKBKGsv1 with a test preparation comprising one or more test compounds; and determining the binding of said test preparation to said IKBKGsv1 polypeptide and to IKBKG isoform polypeptide that is not IKBKGsv1, wherein a test preparation that binds to said IKBKGsv1 polypeptide but does not bind to said IKBKG isoform polypeptide that is not IKBKGsv1 contains a compound that selectively binds said IKBKGsv1 polypeptide. Alternatively, the same method can be performed using IKBKGsv2.1 polypeptide comprising, consisting, or consisting essentially of SEQ ID NO 7. Alternatively, the same method can be performed using IKBKGsv2.2 polypeptide comprising, consisting, or consisting essentially of SEQ ID NO 9. Alternatively, the same method can be performed using IKBKGsv3 polypeptide comprising, consisting, or consisting essentially of SEQ ID NO 11.

In another embodiment of the invention, a method is provided for screening for a compound able to bind to or interact with a IKBKGsv1 protein or a fragment thereof comprising the steps of: expressing a IKBKGsv1 polypeptide comprising SEQ ID NO 5 or a fragment thereof from a recombinant nucleic acid; providing to said polypeptide a labeled IKBKG ligand that binds to said polypeptide and a test preparation comprising one or more compounds; and measuring the effect of said test preparation on binding of said labeled IKBKG ligand to said polypeptide, wherein a test preparation that alters the binding of said labeled IKBKG ligand to said polypeptide contains a compound that binds to or interacts with said polypeptide. In an alternative embodiment, the method is performed using IKBKGsv2.1 polypeptide comprising, consisting, or consisting essentially of SEQ ID NO 7 or a fragment thereof. In an alternative embodiment, the method is performed using IKBKGsv2.2 polypeptide comprising, consisting, or consisting essentially of SEQ ID NO 9 or a fragment thereof. In an alternative embodiment, the method is performed using IKBKGsv3 polypeptide comprising, consisting, or consisting essentially of SEQ ID NO 11 or a fragment thereof.

Other features and advantages of the present invention are apparent from the additional descriptions provided herein, including the different examples. The provided examples illustrate different components and methodology useful in practicing the present invention. The examples do not limit the claimed invention. Based on the present disclosure the skilled artisan can identify and employ other components and methodology useful for practicing the present invention.

Definitions

Unless defined otherwise, all technical and scientific terms used herein have the meaning commonly understood by one of ordinary skill in the art to which this invention belongs.

As used herein, “IKBKG” refers to an inhibitor of kappa light polypeptide gene enhancer in B-cells, kinase of, gamma protein (NP_{—}003630). In contrast, reference to an IKBKG isoform, includes NP_{—}003630 and other polypeptide isoform variants of IKBKG.

As used herein, “IKBKGsv1”, “IKBKGsv2.1”, “IKBKGsv2.2” and “IKBKGsv3” refer to splice variant isoforms of human IKBKG protein, wherein the splice variants have the amino acid sequence set forth in SEQ ID NO 5 (for IKBKGsv1), SEQ ID NO 7 (for IKBKGsv2.1), SEQ ID NO 9 (for IKBKGsv2.2), and SEQ ID NO 11 (for IKBKGsv3).

As used herein, “IKBKG” refers to polynucleotides encoding IKBKG.

As used herein, “IKBKGsv2” refers to polynucleotides that are identical to IKBKG encoding polynucleotides, except that the sequences represented by exons 4 and 5 of the IKBKG messenger RNA are not present in IKBKGsv2.

As used herein, “IKBKGsv1” refers to polynucleotides encoding IKBKGsv1 having an amino acid sequence set forth in SEQ ID NO 5. As used herein, “IKBKGsv2.1” refers to polynucleotides encoding IKBKGsv2.1 having an amino acid sequence set forth in SEQ ID NO 7. As used herein, “IKBKGsv2.2” refers to polynucleotides encoding IKBKGsv2.2 having an amino acid sequence set forth in SEQ ID NO 9. As used herein, “IKBKGsv3” refers to polynucleotides encoding IKBKGsv3 having an amino acid sequence set forth in SEQ ID NO 11.

As used herein, an “isolated nucleic acid” is a nucleic acid molecule that exists in a physical form that is nonidentical to any nucleic acid molecule of identical sequence as found in nature; “isolated” does not require, although it does not prohibit, that the nucleic acid so described has itself been physically removed from its native environment. For example, a nucleic acid can be said to be “isolated” when it includes nucleotides and/or internucleoside bonds not found in nature. When instead composed of natural nucleosides in phosphodiester linkage, a nucleic acid can be said to be “isolated” when it exists at a purity not found in nature, where purity can be adjudged with respect to the presence of nucleic acids of other sequence, with respect to the presence of proteins, with respect to the presence of lipids, or with respect the presence of any other component of a biological cell, or when the nucleic acid lacks sequence that flanks an otherwise identical sequence in an organism's genome, or when the nucleic acid possesses sequence not identically present in nature. As so defined, “isolated nucleic acid” includes nucleic acids integrated into a host cell chromosome at a heterologous site, recombinant fusions of a native fragment to a heterologous sequence, recombinant vectors present as episomes or as integrated into a host cell chromosome.

A “purified nucleic acid” represents at least 10% of the total nucleic acid present in a sample or preparation. In preferred embodiments, the purified nucleic acid represents at least about 50%, at least about 75%, or at least about 95% of the total nucleic acid in a isolated nucleic acid sample or preparation. Reference to “purified nucleic acid” does not require that the nucleic acid has undergone any purification and may include, for example, chemically synthesized nucleic acid that has not been purified.

The phrases “isolated protein”, “isolated polypeptide”, “isolated peptide” and “isolated oligopeptide” refer to a protein (or respectively to a polypeptide, peptide, or oligopeptide) that is nonidentical to any protein molecule of identical amino acid sequence as found in nature; “isolated” does not require, although it does not prohibit, that the protein so described has itself been physically removed from its native environment. For example, a protein can be said to be “isolated” when it includes amino acid analogues or derivatives not found in nature, or includes linkages other than standard peptide bonds. When instead composed entirely of natural amino acids linked by peptide bonds, a protein can be said to be “isolated” when it exists at a purity not found in nature—where purity can be adjudged with respect to the presence of proteins of other sequence, with respect to the presence of non-protein compounds, such as nucleic acids, lipids, or other components of a biological cell, or when it exists in a composition not found in nature, such as in a host cell that does not naturally express that protein.

As used herein, a “purified polypeptide” (equally, a purified protein, peptide, or oligopeptide) represents at least 10% of the total protein present in a sample or preparation, as measured on a weight basis with respect to total protein in a composition. In preferred embodiments, the purified polypeptide represents at least about 50%, at least about 75%, or at least about 95% of the total protein in a sample or preparation. A “substantially purified protein” (equally, a substantially purified polypeptide, peptide, or oligopeptide) is an isolated protein, as above described, present at a concentration of at least 70%, as measured on a weight basis with respect to total protein in a composition. Reference to “purified polypeptide” does not require that the polypeptide has undergone any purification and may include, for example, chemically synthesized polypeptide that has not been purified.

As used herein, the term “antibody” refers to a polypeptide, at least a portion of which is encoded by at least one immunoglobulin gene, or fragment thereof, and that can bind specifically to a desired target molecule. The term includes naturally-occurring forms, as well as fragments and derivatives. Fragments within the scope of the term “antibody” include those produced by digestion with various proteases, those produced by chemical cleavage and/or chemical dissociation, and those produced recombinantly, so long as the fragment remains capable of specific binding to a target molecule. Among such fragments are Fab, Fab′, Fv, F(ab)′₂, and single chain Fv (scFv) fragments. Derivatives within the scope of the term include antibodies (or fragments thereof) that have been modified in sequence, but remain capable of specific binding to a target molecule, including: interspecies chimeric and humanized antibodies; antibody fusions; heteromeric antibody complexes and antibody fusions, such as diabodies (bispecific antibodies), single-chain diabodies, and intrabodies (see, e.g., Marasco (ed.), Intracellular Antibodies: Research and Disease Applications, Springer-Verlag New York, Inc. (1998) (ISBN: 3540641513). As used herein, antibodies can be produced by any known technique, including harvest from cell culture of native B lymphocytes, harvest from culture of hybridomas, recombinant expression systems, and phage display.

As used herein, a “purified antibody preparation” is a preparation where at least 10% of the antibodies present bind to the target ligand. In preferred embodiments, antibodies binding to the target ligand represent at least about 50%, at least about 75%, or at least about 95% of the total antibodies present. Reference to “purified antibody preparation” does not require that the antibodies in the preparation have undergone any purification.

As used herein, “specific binding” refers to the ability of two molecular species concurrently present in a heterogeneous (inhomogeneous) sample to bind to one another in preference to binding to other molecular species in the sample. Typically, a specific binding interaction will discriminate over adventitious binding interactions in the reaction by at least two-fold, more typically by at least 10-fold, often at least 100-fold; when used to detect analyte, specific binding is sufficiently discriminatory when determinative of the presence of the analyte in a heterogeneous (inhomogeneous) sample. Typically, the affinity or avidity of a specific binding reaction is least about 1 μM.

The term “antisense”, as used herein, refers to a nucleic acid molecule sufficiently complementary in sequence, and sufficiently long in that complementary sequence, as to hybridize under intracellular conditions to (i) a target mRNA transcript or (ii) the genomic DNA strand complementary to that transcribed to produce the target mRNA transcript.

The term “subject”, as used herein refers to an organism and to cells or tissues derived therefrom. For example the organism may be an animal, including but not limited to animals such as cows, pigs, horses, chickens, cats, dogs, etc., and is usually a mammal, and most commonly human.

### DETAILED DESCRIPTION OF THE INVENTION

This section presents a detailed description of the present invention and its applications. This description is by way of several exemplary illustrations, in increasing detail and specificity, of the general methods of this invention. These examples are non-limiting, and related variants that will be apparent to one of skill in the art are intended to be encompassed by the appended claims.

The present invention relates to the nucleic acid sequences encoding human IKBKGsv1, IKBKGsv2.1, IKBKGsv2.2, and IKBKGsv3 that are alternatively spliced isoforms of IKBKG, and to the amino acid sequences encoding these proteins. SEQ ID NO 4, SEQ ID NO 6, SEQ ID NO 8 and SEQ ID NO 10 are polynucleotide sequences representing exemplary open reading frames that encode the IKBKGsv1, IKBKGsv2.1, IKBKGsv2.2, and IKBKGsv3 proteins, respectively. SEQ ID NO 5 shows the polypeptide sequence of IKBKGsv1. SEQ ID NO 7 shows the polypeptide sequence of IKBKGsv2.1. SEQ ID NO 9 shows the polypeptide sequence of IKBKGsv2.2. SEQ ID NO 11 shows the polypeptide sequence of IKBKGsv3.

IKBKGsv1, IKBKGsv2.1, IKBKGsv2.2, and IKBKGsv3 polynucleotide sequences encoding IKBKGsv1, IKBKGsv2.1, IKBKGsv2.2, and IKBKGsv3 proteins, as exemplified and enabled herein include a number of specific, substantial and credible utilities. For example, IKBKGsv1, IKBKGsv2.1, IKBKGsv2.2, and IKBKGsv3 encoding nucleic acids were identified in a mRNA sample obtained from a human source (see Example 1). Such nucleic acids can be used as hybridization probes to distinguish between cells that produce IKBKGsv1, IKBKGsv2.1, IKBKGsv2.2, and IKBKGsv3 transcripts from human or non-human cells (including bacteria) that do not produce such transcripts. Similarly, antibodies specific for IKBKGsv1, IKBKGsv2.1, IKBKGsv2.2, or IKBKGsv3 can be used to distinguish between cells that express IKBKGsv1, IKBKGsv2.1, IKBKGsv2.2, or IKBKGsv3 from human or non-human cells (including bacteria) that do not express IKBKGsv1, IKBKGsv2.1, IKBKGsv2.2, or IKBKGsv3.

IKBKG is an important drug target for the management of immune function and inflammation responses, as well as diseases such as rheumatoid arthritis, lupus, HIV-AIDS, influenza, and cancer (Baldwin, A. S., 1996, Annu. Rev. Immunol. 14, 649-681; May, et. al., 2000, Science 289, 1550-1554). Given the potential importance of IKBKG activity to the therapeutic management of a wide array of diseases, it is of value to identify IKBKG isoforms and identify IKBKG-ligand compounds that are isoform specific, as well as compounds that are effective ligands for two or more different IKBKG isoforms. In particular, it may be important to identify compounds that are effective inhibitors of a specific IKBKG isoform activity, yet does not bind to or interact with a plurality of different IKBKG isoforms. Compounds that bind to or interact with multiple IKBKG isoforms may require higher drug doses to saturate multiple IKBKG-isoform binding sites and thereby result in a greater likelihood of secondary non-therapeutic side effects. Furthermore, biological effects could also be caused by the interactions of a drug with the IKBKGsv1, IKBKGsv2.1, IKBKGsv2.2, or IKBKGsv3 isoforms specifically. For the foregoing reasons, IKBKGsv1, IKBKGsv2.1, IKBKGsv2.2, and IKBKGsv3 proteins represent useful compound binding targets and have utility in the identification of new IKBKG-ligands exhibiting a preferred specificity profile and having greater efficacy for their intended use.

In some embodiments, IKBKGsv1, IKBKGsv2.1, IKBKGsv2.2, and IKBKGsv3 activity is modulated by a ligand compound to achieve one or more of the following: prevent or reduce the risk of occurrence, or recurrence of rheumatoid arthritis, septic shock, lupus, HIV-AIDS, viral infections, and cancer. Compounds that treat cancers are particularly important because of the cause-and-effect relationship between cancers and mortality (National Cancer Institute's Cancer Mortality Rates Registry, http://www3.cancer.gov/atlasplus/charts.html, last visited Dec. 31, 2002).

Compounds modulating IKBKGsv1, IKBKGsv2.1, IKBKGsv2.2, or IKBKGsv3 include agonists, antagonists, and allosteric modulators. While not wishing to be limited to any particular theory of therapeutic efficacy, generally, but not always, IKBKGsv1, IKBKGsv2.1, IKBKGsv2.2, or IKBKGsv3 compounds will be used to inhibit release of NF-kappa-B to the nucleus. The inhibition of IKBKG has been shown to have therapeutic effects in the treatment of acute inflammation in model mouse systems (May, et. al., 2000, Science 289, 1550-1554). Inhibitors of IKBKG achieve clinical efficacy by a number of known and unknown mechanisms. It is hypothesized that inhibition of IKBKG will prevent the formation of the I-kappa-B kinase complex and the subsequent cascade of events leading to the release of NF-kappa-B to the nucleus. This is because IKBKG is essential for the formation of the I-kappa-B kinase complex, which is responsible for the phosphorylation of 1-kappa-B and subsequent release of NF-kappa-B to the nucleus, activating a gene response, (May, 2000). It is further hypothesized that inhibition of IKBKG will allow maintenance of low levels of NF-kappa-B basal activity, thereby reducing potential toxic side effects (May, 2000). Therefore, agents that modulate IKBKG activity may be used to achieve a therapeutic benefit for any disease or condition due to, or exacerbated by, abnormal levels of NF-kappa-B protein or its activity.

IKBKGsv1, IKBKGsv2.1, IKBKGsv2.2, or IKBKGsv3 activity can also be affected by modulating the cellular abundance of transcripts encoding IKBKGsv1, IKBKGsv2.1, IKBKGsv2.2, or IKBKGsv3, respectively. Compounds modulating the abundance of transcripts encoding IKBKGsv1, IKBKGsv2.1, IKBKGsv2.2, or IKBKGsv3 include a cloned polynucleotide encoding IKBKGsv1, IKBKGsv2.1, IKBKGsv2.2, or IKBKGsv3, respectively, that can express IKBKGsv1, IKBKGsv2.1, IKBKGsv2.2, or IKBKGsv3 in vivo, antisense nucleic acids targeted to IKBKGsv1, IKBKGsv2.1, IKBKGsv2.2, or IKBKGsv3 transcripts, and enzymatic nucleic acids, such as ribozymes and RNAi (siRNA and shRNA), targeted to IKBKGsv1, IKBKGsv2.1, IKBKGsv2.2, or IKBKGsv3 transcripts.

In some embodiments, IKBKGsv1, IKBKGsv2.1, IKBKGsv2.2, or IKBKGsv3 activity is modulated to achieve a therapeutic effect upon diseases in which regulation of NF-kappa-B is desirable. For example, rheumatoid arthritis and lupus may be treated by modulating IKBKGsv1, IKBKGsv2.1, IKBKGsv2.2, or IKBKGsv3 activities to inhibit activation of genes involved in inflammation responses. In other embodiments, HIV-AIDS and other viral infections may be treated by inhibiting the activation of genes involved in viral replication. In other embodiments, cancer may be treated by modulating IKBKGsv1, IKBKGsv2.1, IKBKGsv2.2, or IKBKGsv3 to inhibit genes involved in oncogenesis.

IKBKGsv1, IKBKGsv2.1, IKBKGsv2.2, and IKBKGsv3 Nucleic Acids

IKBKGsv1 nucleic acids contain regions that encode for polypeptides comprising, consisting, or consisting essentially of SEQ ID NO 5. IKBKGsv2.1 nucleic acids contain regions that encode for polypeptides comprising, consisting, or consisting essentially of SEQ ID NO 7. IKBKGsv2.2 nucleic acids contain regions that encode for polypeptides comprising, consisting, or consisting essentially of SEQ ID NO 9. IKBKGsv3 nucleic acids contain regions that encode for polypeptides comprising, consisting, or consisting essentially of SEQ ID NO 11. The IKBKGsv1, IKBKGsv2.1, IKBKGsv2.2, and IKBKGsv3 nucleic acids have a variety of uses, such as use as a hybridization probe or PCR primer to identify the presence of IKBKGsv1, IKBKGsv2.1, IKBKGsv2.2, or IKBKGsv3 nucleic acids, respectively; use as a hybridization probe or PCR primer to identify nucleic acids encoding for proteins related to IKBKGsv1, IKBKGsv2.1, IKBKGsv2.2, or IKBKGsv3, respectively; and/or use for recombinant expression of IKBKGsv1, IKBKGsv2.1, IKBKGsv2.2, or IKBKGsv3 polypeptides, respectively. In particular, IKBKGsv1 polynucleotides do not have the polynucleotide region that comprises exon 5 of the IKBKG gene. IKBKGsv2.1 polynucleotides do not have the polynucleotide regions that comprise exons 4 and 5 of the IKBKG gene. IKBKGsv2.2 polynucleotides do not have the polynucleotide regions that comprise exons 1, 2, 3, 4, 5, and 6, and the first 3 nucleotides of exon 7 of the IKBKG gene. IKBKGsv3 polynucleotides do not have the polynucleotide regions that comprise exons 3, 4, 5, and 6 of the IKBKG gene.

Regions in IKBKGsv1, IKBKGsv2.1, IKBKGsv2.2, or IKBKGsv3 nucleic acid that do not encode for IKBKGsv1, IKBKGsv2.1, IKBKGsv2.2, or IKBKGsv3, or are not found in SEQ ID NO 4, SEQ ID NO 6, SEQ ID NO 8 or SEQ ID NO 10, if present, are preferably chosen to achieve a particular purpose. Examples of additional regions that can be used to achieve a particular purpose include: a stop codon that is effective at protein synthesis termination; capture regions that can be used as part of an ELISA sandwich assay; reporter regions that can be probed to indicate the presence of the nucleic acid; expression vector regions; and regions encoding for other polypeptides.

The guidance provided in the present application can be used to obtain the nucleic acid sequence encoding IKBKGsv1, IKBKGsv2.1, IKBKGsv2.2, or IKBKGsv3 related proteins from different sources. Obtaining nucleic acids IKBKGsv1, IKBKGsv2.1, IKBKGsv2.2, or IKBKGsv3 related proteins from different sources is facilitated by using sets of degenerative probes and primers and the proper selection of hybridization conditions. Sets of degenerative probes and primers are produced taking into account the degeneracy of the genetic code. Adjusting hybridization conditions is useful for controlling probe or primer specificity to allow for hybridization to nucleic acids having similar sequences.

Techniques employed for hybridization detection and PCR cloning are well known in the art. Nucleic acid detection techniques are described, for example, in Sambrook, et al., in Molecular Cloning, A Laboratory Manual, 2^{nd} Edition, Cold Spring Harbor Laboratory Press, 1989. PCR cloning techniques are described, for example, in White, Methods in Molecular Cloning, volume 67, Humana Press, 1997.

IKBKGsv1, IKBKGsv2.1, IKBKGsv2.2, or IKBKGsv3 probes and primers can be used to screen nucleic acid libraries containing, for example, cDNA. Such libraries are commercially available, and can be produced using techniques such as those described in Ausubel, Current Protocols in Molecular Biology, John Wiley, 1987-1998.

Starting with a particular amino acid sequence and the known degeneracy of the genetic code, a large number of different encoding nucleic acid sequences can be obtained. The degeneracy of the genetic code arises because almost all amino acids are encoded for by different combinations of nucleotide triplets or “codons”. The translation of a particular codon into a particular amino acid is well known in the art (see, e.g., Lewin GENES IV, p. 119, Oxford University Press, 1990). Amino acids are encoded for by codons as follows:

A=Ala=Alanine: codons GCA, GCC, GCG, GCU

C=Cys=Cysteine: codons UGC, UGU

D=Asp=Aspartic acid: codons GAC, GAU

E=Glu=Glutamic acid: codons GAA, GAG

F=Phe=Phenylalanine: codons UUC, UUU

G=Gly=Glycine: codons GGA, GGC, GGG, GGU

H=His=Histidine: codons CAC, CAU

I=Ile=Isoleucine: codons AUA, AUC, AUU

K=Lys=Lysine: codons AAA, AAG

L=Leu=Leucine: codons UUA, UUG, CUA, CUC, CUG, CUU

M=Met=Methionine: codon AUG

N=Asn=Asparagine: codons AAC, AAU

P=Pro=Proline: codons CCA, CCC, CCG, CCU

Q=Gln=Glutamine: codons CAA, CAG

R=Arg=Arginine: codons AGA, AGG, CGA, CGC, CGG, CGU

S=Ser=Serine: codons AGC, AGU, UCA, UCC, UCG, UCU

T=Thr=Threonine: codons ACA, ACC, ACG, ACU

V=Val=Valine: codons GUA, GUC, GUG, GUU

W=Trp=Tryptophan: codon UGG

Y=Tyr=Tyrosine: codons UAC, UAU

Nucleic acid having a desired sequence can be synthesized using chemical and biochemical techniques. Examples of chemical techniques are described in Ausubel, Current Protocols in Molecular Biology, John Wiley, 1987-1998, and Sambrook et al., in Molecular Cloning, A Laboratory Manual, 2^{nd} Edition, Cold Spring Harbor Laboratory Press, 1989. In addition, long polynucleotides of a specified nucleotide sequence can be ordered from commercial vendors, such as Blue Heron Biotechnology, Inc. (Bothell, Wash.).

Biochemical synthesis techniques involve the use of a nucleic acid template and appropriate enzymes such as DNA and/or RNA polymerases. Examples of such techniques include in vitro amplification techniques such as PCR and transcription based amplification, and in vivo nucleic acid replication. Examples of suitable techniques are provided by Ausubel, Current Protocols in Molecular Biology, John Wiley, 1987-1998, Sambrook et al., in Molecular Cloning, A Laboratory Manual, 2^{nd} Edition, Cold Spring Harbor Laboratory Press, 1989, and U.S. Pat. No. 5,480,784.

IKBKGsv1, IKBKGsv2.1, and IKBKGsv3 Probes

Probes for IKBKGsv1, IKBKGsv2.1, or IKBKGsv3 contain a region that can specifically hybridize to IKBKGsv1, IKBKGsv2.1, or IKBKGsv3 target nucleic acids, respectively, under appropriate hybridization conditions and can distinguish IKBKGsv1, IKBKGsv2.1, or IKBKGsv3 nucleic acids from each other and from non-target nucleic acids, in particular IKBKG polynucleotides containing exons 3, 4, 5, and 6. Probes for IKBKGsv1, IKBKGsv2.1, or IKBKGsv3 can also contain nucleic acid regions that are not complementary to IKBKGsv1, IKBKGsv2.1, or IKBKGsv3 nucleic acids.

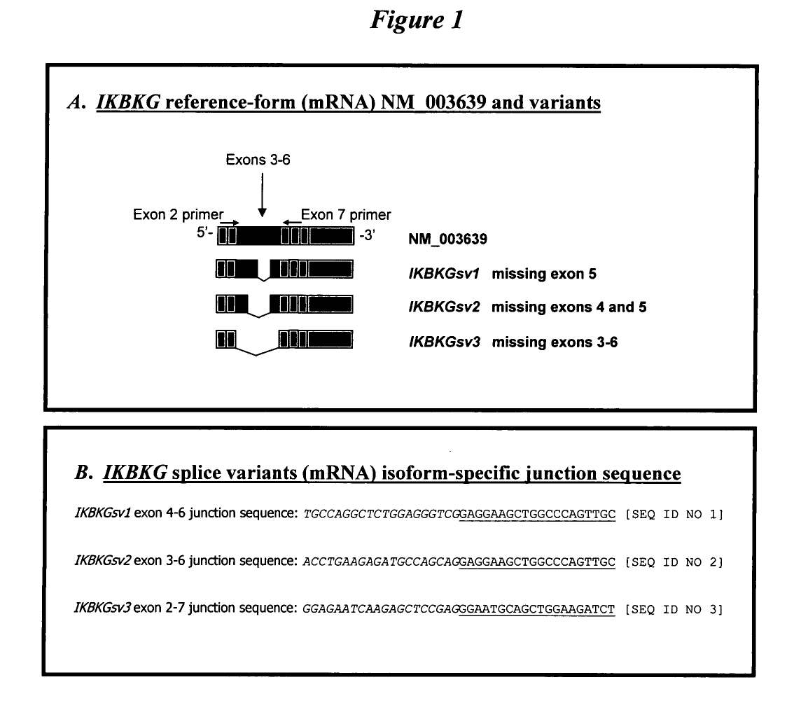

In embodiments where, for example, IKBKGsv1, IKBKGsv2.1, or IKBKGsv3 polynucleotide probes are used in hybridization assays to specifically detect the presence of IKBKGsv1, IKBKGsv2.1, or IKBKGsv3 polynucleotides in samples, the IKBKGsv1, IKBKGsv2.1, or IKBKGsv3 polynucleotides comprise at least 20 nucleotides of the IKBKGsv1, IKBKGsv2.1, or IKBKGsv3 sequence that correspond to the respective novel exon junction polynucleotide regions. In particular, for detection of IKBKGsv1, the probe comprises at least 20 nucleotides of the IKBKGsv1 sequence that corresponds to an exon junction polynucleotide created by the alternative splicing of exon 4 to exon 6 of the primary transcript of the IKBKG gene (see FIGS. 1A and 1B). For example, the polynucleotide sequence: 5′ TGGAGGGTCGGAGGAAGCTG 3′ [SEQ ID NO: 12] represents one embodiment of such an inventive IKBKGsv1 polynucleotide wherein a first 10 nucleotides region is complementary and hybridizable to the 3′ end of exon 4 of the IKBKG gene and a second 10 nucleotide region is complementary and hybridizable to the 5′ end of exon 6 of the IKBKG gene (see FIG. 1B).

In another embodiment, for detection of IKBKGsv2.1, the probe comprises at least 20 nucleotides of the IKBKGsv2.1 sequence that corresponds to an exon junction polynucleotide created by the alternative splicing of exon 3 to exon 6 of the primary transcript of the IKBKG gene (see FIGS. 1A and 1B). For example, the polynucleotide sequence: 5′ ATGCCAGCAGGAGGAAGCTG 3′ [SEQ ID NO: 13] represents one embodiment of such an inventive IKBKGsv2.1 polynucleotide wherein a first 10 nucleotides region is complementary and hybridizable to the 3′ end of exon 3 of the IKBKG gene and a second 10 nucleotide region is complementary and hybridizable to the 5′ end of exon 6 of the IKBKG gene (see FIG. 1B).

In another embodiment, for detection of IKBKGsv3, the probe comprises at least 20 nucleotides of the IKBKGsv3 sequence that corresponds to an exon junction polynucleotide created by the alternative splicing of exon 2 to exon 7 of the primary transcript of the IKBKG gene (see FIGS. 1A and 1B). For example, the polynucleotide sequence: 5′ GAGCTCCGAGGGAATGCAGC 3′ [SEQ ID NO: 14] represents one embodiment of such an inventive IKBKGsv3 polynucleotide wherein a first 10 nucleotides region is complementary and hybridizable to the 3′ end of exon 2 of the IKBKG gene and a second 10 nucleotide region is complementary and hybridizable to the 5′ end of exon 7 of the IKBKG gene (see FIG. 1B).

In some embodiments, the first 20 nucleotides of a IKBKGsv1 probe comprise a first continuous region of 5 to 15 nucleotides that is complementary and hybridizable to the 3′ end of exon 4 and a second continuous region of 5 to 15 nucleotides that is complementary and hybridizable to the 5′ end of exon 6. In some embodiments, the first 20 nucleotides of a IKBKGsv2.1 probe comprise a first continuous region of 5 to 15 nucleotides that is complementary and hybridizable to the 3′ end of exon 3 and a second continuous region of 5 to 15 nucleotides that is complementary and hybridizable to the 5′ end of exon 6. In some embodiments, the first 20 nucleotides of a IKBKGsv3 probe comprise a first continuous region of 5 to 15 nucleotides that is complementary and hybridizable to the 3′ end of exon 2 and a second continuous region of 5 to 15 nucleotides that is complementary and hybridizable to the 5′ end of exon 7.

In other embodiments, the IKBKGsv1, IKBKGsv2.1, or IKBKGsv3 polynucleotide comprise at least 40, 60, 80 or 100 nucleotides of the IKBKGsv1, IKBKGsv2.1, or IKBKGsv3 sequence, respectively, that correspond to a junction polynucleotide region created by the alternative splicing of exon 4 to exon 6 in the case of IKBKGsv1, that correspond to a junction polynucleotide region created by the alternative splicing of exon 3 to exon 6 in the case of IKBKGsv2.1, or in the case of IKBKGsv3, by the alternative splicing of exon 2 to exon 7 of the primary transcript of the IKBKG gene. In embodiments involving IKBKGsv1, the IKBKGsv1 polynucleotide is selected to comprise a first continuous region of at least 5 to 15 nucleotides that is complementary and hybridizable to the 3′ end of exon 4 and a second continuous region of at least 5 to 15 nucleotides that is complementary and hybridizable to the 5′ end of exon 6. Similarly, in embodiments involving IKBKGsv2.1, the IKBKGsv2.1 polynucleotide is selected to comprise a first continuous region of at least 5 to 15 nucleotides that is complementary and hybridizable to the 3′ end of exon 3 and a second continuous region of at least 5 to 15 nucleotides that is complementary and hybridizable to the 5′ end of exon 6. Similarly, in embodiments involving IKBKGsv3, the IKBKGsv3 polynucleotide is selected to comprise a first continuous region of at least 5 to 15 nucleotides that is complementary and hybridizable to the 3′ end of exon 2 and a second continuous region of at least 5 to 15 nucleotides that is complementary and hybridizable to the 5′ end of exon 7. As will be apparent to a person of skill in the art, a large number of different polynucleotide sequences from the region of the exon 4 to exon 6 splice junction, the exon 3 to exon 6 splice junction, and the exon 2 to exon 7 splice junction may be selected which will, under appropriate hybridization conditions, have the capacity to detectably hybridize to IKBKGsv1, IKBKGsv2.1, or IKBKGsv3 polynucleotides, respectively, and yet will hybridize to a much less extent or not at all to IKBKG isoform polynucleotides wherein exon 4 is not spliced to exon 6, wherein exon 3 is not spliced to exon 6, or wherein exon 2 is not spliced to exon 7, respectively.

Preferably, non-complementary nucleic acid that is present has a particular purpose such as being a reporter sequence or being a capture sequence. However, additional nucleic acid need not have a particular purpose as long as the additional nucleic acid does not prevent the IKBKGsv1, IKBKGsv2.1, or IKBKGsv3 nucleic acid from distinguishing between target polynucleotides, e.g., IKBKGsv1, IKBKGsv2.1, or IKBKGsv3 polynucleotides, and non-target polynucleotides, including, but not limited to IKBKG polynucleotides not comprising the exon 4 to exon 6 splice junction, comprising the exon 3 to exon 6 splice junction or the exon 2 to exon 7 splice junctions found in IKBKGsv1, IKBKGsv2.1, or IKBKGsv3, respectively.

Hybridization occurs through complementary nucleotide bases. Hybridization conditions determine whether two molecules, or regions, have sufficiently strong interactions with each other to form a stable hybrid.

The degree of interaction between two molecules that hybridize together is reflected by the melting temperature (Tₘ) of the produced hybrid. The higher the Tₘ the stronger the interactions and the more stable the hybrid. Tₘ is effected by different factors well known in the art such as the degree of complementarity, the type of complementary bases present (e.g., A-T hybridization versus G-C hybridization), the presence of modified nucleic acid, and solution components (e.g., Sambrook, et al., in Molecular Cloning, A Laboratory Manual, 2^{nd} Edition, Cold Spring Harbor Laboratory Press, 1989).

Stable hybrids are formed when the Tₘ of a hybrid is greater than the temperature employed under a particular set of hybridization assay conditions. The degree of specificity of a probe can be varied by adjusting the hybridization stringency conditions. Detecting probe hybridization is facilitated through the use of a detectable label. Examples of detectable labels include luminescent, enzymatic, and radioactive labels.

Examples of stringency conditions are provided in Sambrook, et al., in Molecular Cloning, A Laboratory Manual, 2^{nd} Edition, Cold Spring Harbor Laboratory Press, 1989. An example of high stringency conditions is as follows: Prehybridization of filters containing DNA is carried out for 2 hours to overnight at 65° C. in buffer composed of 6×SSC, 5× Denhardt's solution, and 100 μg/ml denatured salmon sperm DNA. Filters are hybridized for 12 to 48 hours at 65° C. in prehybridization mixture containing 100 μg/ml denatured salmon sperm DNA and 5-20×10⁶ cpm of ³²P-labeled probe. Filter washing is done at 37° C. for 1 hour in a solution containing 2×SSC, 0.1% SDS. This is followed by a wash in 0.1×SSC, 0.1% SDS at 50° C. for 45 minutes before autoradiography. Other procedures using conditions of high stringency would include, for example, either a hybridization step carried out in 5×SSC, 5× Denhardt's solution, 50% formamide at 42° C. for 12 to 48 hours or a washing step carried out in 0.2×SSPE, 0.2% SDS at 65° C. for 30 to 60 minutes.

Recombinant Expression

IKBKGsv1, IKBKGsv2.1, IKBKGsv2.2, or IKBKGsv3 polynucleotides, such as those comprising SEQ ID NO 4, SEQ ID NO 6, SEQ ID NO 8, or SEQ ID NO 10, respectively, can be used to make IKBKGsv1, IKBKGsv2.1, IKBKGsv2.2, or IKBKGsv3 polypeptides, respectively. In particular, IKBKGsv1, IKBKGsv2.1, IKBKGsv2.2, or IKBKGsv3 polypeptides can be expressed from recombinant nucleic acids in a suitable host or in vitro using a translation system. Recombinantly expressed IKBKGsv1, IKBKGsv2.1, IKBKGsv2.2, or IKBKGsv3 polypeptides can be used, for example, in assays to screen for compounds that bind IKBKGsv1, IKBKGsv2.1, IKBKGsv2.2, or IKBKGsv3, respectively. Alternatively, IKBKGsv1, IKBKGsv2.1, IKBKGsv2.2, or IKBKGsv3 polypeptides can also be used to screen for compounds that bind to one or more IKBKG isoforms, but do not bind to IKBKGsv1, IKBKGsv2.1, IKBKGsv2.2, or IKBKGsv3, respectively.

In some embodiments, expression is achieved in a host cell using an expression vector. An expression vector contains recombinant nucleic acid encoding a polypeptide along with regulatory elements for proper transcription and processing. The regulatory elements that may be present include those naturally associated with the recombinant nucleic acid and exogenous regulatory elements not naturally associated with the recombinant nucleic acid. Exogenous regulatory elements such as an exogenous promoter can be useful for expressing recombinant nucleic acid in a particular host.

Generally, the regulatory elements that are present in an expression vector include a transcriptional promoter, a ribosome binding site, a terminator, and an optionally present operator. Another preferred element is a polyadenylation signal providing for processing in eukaryotic cells. Preferably, an expression vector also contains an origin of replication for autonomous replication in a host cell, a selectable marker, a limited number of useful restriction enzyme sites, and a potential for high copy number. Examples of expression vectors are cloning vectors, modified cloning vectors, and specifically designed plasmids and viruses.

Expression vectors providing suitable levels of polypeptide expression in different hosts are well known in the art. Mammalian expression vectors well known in the art include, but are not restricted to, pcDNA3 (Invitrogen, Carlsbad Calif.), pSecTag2 (Invitrogen), pMC1neo (Stratagene, La Jolla Calif.), pXT1 (Stratagene), pSG5 (Stratagene), pCMVLac1 (Stratagene), pCI-neo (Promega), EBO-pSV2-neo (ATCC 37593), pBPV-1(8-2) (ATCC 37110), pdBPV-MMTneo(342-12) (ATCC 37224), pRSVgpt (ATCC 37199), pRSVneo (ATCC 37198), pSV2-dhfr (ATCC 37146) and pUCTag (ATCC 37460), and Bacterial expression vectors well known in the art include pET11a (Novagen), pBluescript SK (Stratagene, La Jolla), pQE-9 (Qiagen Inc., Valencia), lambda gt11 (Invitrogen), pcDNAII (Invitrogen), and pKK223-3 (Pharmacia). Fungal cell expression vectors well known in the art include pPICZ (Invitrogen) and pYES2 (Invitrogen), Pichia expression vector (Invitrogen). Insect cell expression vectors well known in the art include Blue Bac III (Invitrogen), pBacPAK8 (CLONTECH, Inc., Palo Alto) and PfastBacHT (Invitrogen, Carlsbad).

Recombinant host cells may be prokaryotic or eukaryotic. Examples of recombinant host cells include the following: bacteria such as E. coli; fungal cells such as yeast; mammalian cells such as human, bovine, porcine, monkey and rodent; and insect cells such as Drosophila and silkworm derived cell lines. Commercially available mammalian cell lines include L cells L-M(TK⁻) (ATCC CCL 1.3), L cells L-M (ATCC CCL 1.2), 293 (ATCC CRL 1573), Raji (ATCC CCL 86), CV-1 (ATCC CCL 70), COS-1 (ATCC CRL 1650), COS-7 (ATCC CRL 1651), CHO-KI (ATCC CCL 61), 3T3 (ATCC CCL 92), NIH/3T3 (ATCC CRL 1658), HeLa (ATCC CCL 2), C1271 (ATCC CRL 1616), BS-C-1 (ATCC CCL 26) MRC-5 (ATCC CCL 171), and HEK 293 cells (ATCC CRL-1573).

To enhance expression in a particular host it may be useful to modify the sequence provided in SEQ ID NO 4, SEQ ID NO 6, SEQ ID NO 8, or SEQ ID NO 10 to take into account codon usage of the host. Codon usage of different organisms are well known in the art (see, Ausubel, Current Protocols in Molecular Biology, John Wiley, 1987-1998, Supplement 33 Appendix 1C).

Expression vectors may be introduced into host cells using standard techniques. Examples of such techniques include transformation, transfection, lipofection, protoplast fusion, and electroporation.

Nucleic acids encoding for a polypeptide can be expressed in a cell without the use of an expression vector employing, for example, synthetic mRNA or native mRNA. Additionally, mRNA can be translated in various cell-free systems such as wheat germ extracts and reticulocyte extracts, as well as in cell based systems, such as frog oocytes. Introduction of mRNA into cell based systems can be achieved, for example, by microinjection or electroporation.

IKBKGsv1, IKBKGsv2.1, IKBKGsv2.2, and IKBKGsv3 Polypeptides

IKBKGsv1 polypeptides contain an amino acid sequence comprising, consisting or consisting essentially of SEQ ID NO 5. IKBKGsv2.1 polypeptides contain an amino acid sequence comprising, consisting or consisting essentially of SEQ ID NO 7. IKBKGsv2.2 polypeptides contain an amino acid sequence comprising, consisting or consisting essentially of SEQ ID NO 9. IKBKGsv3 polypeptides contain an amino acid sequence comprising, consisting or consisting essentially of SEQ ID NO 11. IKBKGsv1, IKBKGsv2.1, IKBKGsv2.2, or IKBKGsv3 polypeptides have a variety of uses, such as providing a marker for the presence of IKBKGsv1, IKBKGsv2.1, IKBKGsv2.2, or IKBKGsv3, respectively; use as an immunogen to produce antibodies binding to IKBKGsv1, IKBKGsv2.1, IKBKGsv2.2, or IKBKGsv3, respectively; use as a target to identify compounds binding selectively to IKBKGsv1, IKBKGsv2.1, IKBKGsv2.2, or IKBKGsv3, respectively; or use in an assay to identify compounds that bind to one or more iosforms of IKBKG but do not bind to or interact with IKBKGsv1, IKBKGsv2.1, IKBKGsv2.2, or IKBKGsv3, respectively.

In chimeric polypeptides containing one or more regions from IKBKGsv1, IKBKGsv2.1, IKBKGsv2.2, or IKBKGsv3 and one or more regions not from IKBKGsv1, IKBKGsv2.1, IKBKGsv2.2, or IKBKGsv3, respectively, the region(s) not from IKBKGsv1, IKBKGsv2.1, IKBKGsv2.2, or IKBKGsv3, respectively, can be used, for example, to achieve a particular purpose or to produce a polypeptide that can substitute for IKBKGsv1, IKBKGsv2.1, IKBKGsv2.2, or IKBKGsv3, or fragments thereof. Particular purposes that can be achieved using chimeric IKBKGsv1, IKBKGsv2.1, IKBKGsv2.2, or IKBKGsv3 polypeptides include providing a marker for IKBKGsv1, IKBKGsv2.1, IKBKGsv2.2, or IKBKGsv3 activity, respectively, enhancing an immune response, and modulating kinase activity of the IKK complex or levels of NF-kappa-B in the nucleus.

Polypeptides can be produced using standard techniques including those involving chemical synthesis and those involving biochemical synthesis. Techniques for chemical synthesis of polypeptides are well known in the art (see e.g., Vincent, in Peptide and Protein Drug Delivery, New York, N.Y., Dekker, 1990).

Biochemical synthesis techniques for polypeptides are also well known in the art. Such techniques employ a nucleic acid template for polypeptide synthesis. The genetic code providing the sequences of nucleic acid triplets coding for particular amino acids is well known in the art (see, e.g., Lewin GENES IV, p. 119, Oxford University Press, 1990). Examples of techniques for introducing nucleic acid into a cell and expressing the nucleic acid to produce protein are provided in references such as Ausubel, Current Protocols in Molecular Biology, John Wiley, 1987-1998, and Sambrook, et al., in Molecular Cloning, A Laboratory Manual, 2^{nd} Edition, Cold Spring Harbor Laboratory Press, 1989.

Functional IKBKGsv1, IKBKGsv2.1, IKBKGsv2.2, and IKBKGsv3

Functional IKBKGsv1, IKBKGsv2.1, IKBKGsv2.2, and IKBKGsv3 are different protein isoforms of IKBKG. The identification of the amino acid and nucleic acid sequences of IKBKGsv1, IKBKGsv2.1, IKBKGsv2.2, or IKBKGsv3 provide tools for obtaining functional proteins related to IKBKGsv1, IKBKGsv2.1, IKBKGsv2.2, or IKBKGsv3, respectively, from other sources, for producing IKBKGsv1, IKBKGsv2.1, IKBKGsv2.2, or IKBKGsv3 chimeric proteins, and for producing functional derivatives of SEQ ID NO 5, SEQ ID NO 7, SEQ ID NO 9, or SEQ ID NO 11.

IKBKGsv1, IKBKGsv2.1, IKBKGsv2.2, or IKBKGsv3 polypeptides can be readily identified and obtained based on their sequence similarity to IKBKGsv1 (SEQ ID NO 5), IKBKGsv2.1 (SEQ ID NO 7), IKBKGsv2.2 (SEQ ID NO 9), or IKBKGsv3 (SEQ ID NO 11), respectively. In particular, IKBKGsv1 lacks the amino acids encoded by exon 5 of the IKBKG gene. The IKBKGsv2.1 polypeptides lack the amino acids encoded by exons 4 and 5 of the IKBKG gene. The deletion of exons 4 and 5 and the splicing of exon 3 to exon 6 of the IKBKG hnRNA transcript results in a shift of the protein reading frame at the exon 3 to exon 6 splice junction, thereby creating an amino terminal peptide region that is unique to the IKBKGsv2.1 polypeptide as compared to other known IKBKG isoforms. The frame shift creates a premature termination codon eighty-five nucleotides downstream of the exon 3/exon 6 splice junction. Thus, the IKBKGsv2.1 polypeptide is also lacking the amino acids encoded by the nucleotides downstream of the premature stop codon. The IKBKGsv2.2 carboxy terminal polypeptide lacks the amino acids encoded by the first 771 nucleotides of the IKBKG gene. Initiation at a downstream AUG of a bicistronic RNA is a fairly common event and can be associated with disease (Meijer and Thomas, 2002 Biochem. J., 367:1-11; Kozak, 2002, Mammalian Genome 13:401-410). The IKBKGsv3 polypeptide lacks the amino acids encoded by exons 3, 4, 5, and 6 of the IKBKG gene. The deletion of exons 3, 4, 5 and 6 results in a reading frame shift, thereby creating amino acids that are unique to the IKBKGsv3 polypeptide. The frame shift creates a premature termination codon seventy-eight nucleotides downstream of (i.e., on the coding strand, 3′ of) the exon 2/exon 7 splice junction. Thus, the IKBKGsv3 polypeptide is also lacking the amino acids encoded by the nucleotides downstream of the premature stop codon.

Both the amino acid and nucleic acid sequences of IKBKGsv1, IKBKGsv2.1, IKBKGsv2.2, or IKBKGsv3 can be used to help identify and obtain IKBKGsv1, IKBKGsv2.1, IKBKGsv2.2, or IKBKGsv3 polypeptides, respectively. For example, SEQ ID NO 1 can be used to produce degenerative nucleic acid probes or primers for identifying and cloning nucleic acid polynucleotides encoding for an IKBKGsv1 polypeptide. In addition, polynucleotides comprising, consisting, or consisting essentially of SEQ ID NO 4 or fragments thereof, can be used under conditions of moderate stringency to identify and clone nucleic acids encoding IKBKGsv1 polypeptides from a variety of different organisms. The same methods can also be performed with polynucleotides comprising, consisting, or consisting essentially of SEQ ID NO 6, SEQ ID NO 8, or SEQ ID NO 10, or fragments thereof, to identify and clone nucleic acids encoding IKBKGsv2.1, IKBKGsv2.2, and IKBKGsv3, respectively.

The use of degenerative probes and moderate stringency conditions for cloning is well known in the art. Examples of such techniques are described by Ausubel, Current Protocols in Molecular Biology, John Wiley, 1987-1998, and Sambrook, et al., in Molecular Cloning, A Laboratory Manual, 2^{nd} Edition, Cold Spring Harbor Laboratory Press, 1989.

Starting with IKBKGsv1, IKBKGsv2.1, IKBKGsv2.2, or IKBKGsv3 obtained from a particular source, derivatives can be produced. Such derivatives include polypeptides with amino acid substitutions, additions and deletions. Changes to IKBKGsv1, IKBKGsv2.1, IKBKGsv2.2, or IKBKGsv3 to produce a derivative having essentially the same properties should be made in a manner not altering the tertiary structure of IKBKGsv1, IKBKGsv2.1, IKBKGsv2.2, or IKBKGsv3, respectively.

Differences in naturally occurring amino acids are due to different R groups. An R group affects different properties of the amino acid such as physical size, charge, and hydrophobicity. Amino acids are can be divided into different groups as follows: neutral and hydrophobic (alanine, valine, leucine, isoleucine, proline, tryptophan, phenylalanine, and methionine); neutral and polar (glycine, serine, threonine, tryosine, cysteine, asparagine, and glutamine); basic (lysine, arginine, and histidine); and acidic (aspartic acid and glutamic acid).

Generally, in substituting different amino acids it is preferable to exchange amino acids having similar properties. Substituting different amino acids within a particular group, such as substituting valine for leucine, arginine for lysine, and asparagine for glutamine are good candidates for not causing a change in polypeptide functioning.

Changes outside of different amino acid groups can also be made. Preferably, such changes are made taking into account the position of the amino acid to be substituted in the polypeptide. For example, arginine can substitute more freely for nonpolar amino acids in the interior of a polypeptide then glutamate because of its long aliphatic side chain (See, Ausubel, Current Protocols in Molecular Biology, John Wiley, 1987-1998, Supplement 33 Appendix 1C).

IKBKGsv1, IKBKGsv2.1, IKBKGsv2.2, and IKBKGsv3 Antibodies

Antibodies recognizing IKBKGsv1, IKBKGsv2.1, IKBKGsv2.2, or IKBKGsv3 can be produced using a polypeptide containing SEQ ID NO 5 in the case of IKBKGsv1, SEQ ID NO 7 in the case of IKBKGsv2.1, SEQ ID NO 9 in the case of IKBKGsv2.2, or SEQ ID NO 11 in the case of IKBKGsv3, respectively, or a fragment thereof, as an immunogen. Preferably, a IKBKGsv1 polypeptide used as an immunogen consists of a polypeptide of SEQ ID NO 5 or a SEQ ID NO 5 fragment having at least 10 contiguous amino acids in length corresponding to the polynucleotide region representing the junction resulting from the splicing of exon 4 to exon 6 of the IKBKG gene. Preferably, a IKBKGsv2.1 polypeptide used as an immunogen consists of a polypeptide derived from SEQ ID NO 7 or a SEQ ID NO 7 fragment, having at least 10 contiguous amino acids in length corresponding to a polynucleotide region representing the junction resulting from the splicing of exon 3 to exon 6 of the IKBKG gene. Preferably, a IKBKGsv2.2 polypeptide used as an immunogen consists of a polypeptide of SEQ ID NO 9 or a SEQ ID NO 9 fragment having at least 10 contiguous amino acids in length corresponding to amino acids, including and downstream of, the amino terminal initiation methionine of IKBKGsv2.2. Preferably, a IKBKGsv3 polypeptide used as an immunogen consists of a polypeptide derived from SEQ ID NO 11 or a SEQ ID NO 11 fragment, having at least 10 contiguous amino acids in length corresponding to a polynucleotide region representing the junction resulting from the splicing of exon 2 to exon 7 of the IKBKG gene.

In some embodiments where, for example, IKBKGsv1 polypeptides are used to develop antibodies that bind specifically to IKBKGsv1 and not to other isoforms of IKBKG, the IKBKGsv1 polypeptides comprise at least 10 amino acids of the IKBKGsv1 polypeptide sequence corresponding to a junction polynucleotide region created by the alternative splicing of exon 4 to exon 6 of the primary transcript of the IKBKG gene (see FIG. 1). For example, the amino acid sequence: amino terminus-QALEGRRKLA-carboxy terminus [SEQ ID NO: 15] represents one embodiment of such an inventive IKBKGsv1 polypeptide wherein a first 5 amino acid region is encoded by nucleotide sequence at the 3′ end of exon 4 of the IKBKG gene and a second 5 amino acid region is encoded by the nucleotide sequence directly after the novel splice junction. Preferably, at least 10 amino acids of the IKBKGsv1 polypeptide comprises a first continuous region of 2 to 8 amino acids that is encoded by nucleotides at the 3′ end of exon 4 and a second continuous region of 2 to 8 amino acids that is encoded by nucleotides at the 5′ end of exon 6.

In other embodiments where, for example, IKBKGsv2.1 polypeptides are used to develop antibodies that bind specifically to IKBKGsv2.1 and not to other IKBKG isoforms, the IKBKGsv2.1 polypeptides comprise at least 10 amino acids of the IKBKGsv2.1 polypeptide sequence corresponding to a junction polynucleotide region created by the alternative splicing of exon 3 to exon 6 of the primary transcript of the IKBKG gene (see FIG. 1). For example, the amino acid sequence: amino terminus-KRCQQEEAGP-carboxy terminus [SEQ ID NO: 16], represents one embodiment of such an inventive IKBKGsv2.1 polypeptide wherein a first 5 amino acid region is encoded by a nucleotide sequence at the 3′ end of exon 3 of the IKBKG gene and a second 5 amino acid region is encoded by a nucleotide sequence directly after the novel splice junction. Preferably, at least 10 amino acids of the IKBKGsv2.1 polypeptide comprises a first continuous region of 2 to 8 amino acids that is encoded by nucleotides at the 3′ end of exon 3 and a second continuous region of 2 to 8 amino acids that is encoded by nucleotides at the 5′ end of exon 6.

In other embodiments where, for example, IKBKGsv2.2 polypeptides are used to develop antibodies that bind specifically to IKBKGsv2.2 and not to other isoforms of IKBKG, the IKBKGsv2.2 polypeptides comprise at least 10 amino acids at the amino terminus of the IKBKGsv2.2 polypeptide sequence having at least 10 contiguous amino acids in length corresponding to amino acids, including and downstream of, the amino terminal initiation methionine of IKBKGsv2.2. For example, the amino acid sequence: amino terminus-MQLEDLKQQL-carboxy terminus [SEQ ID NO: 17], represents one embodiment of such an inventive IKBKGsv2.2 polypeptide wherein a first 10 amino acid region is encoded by a nucleotide sequence starting with the “AUG” codon 3 nucleotides downstream of the 5′ end of exon 7 of the IKBKG gene.

In other embodiments where, for example, IKBKGsv3 polypeptides are used to develop antibodies that bind specifically to IKBKGsv3 and not to other IKBKG isoforms, the IKBKGsv3 polypeptides comprise at least 10 amino acids of the IKBKGsv3 polypeptide sequence corresponding to a junction polynucleotide region created by the alternative splicing of exon 2 to exon 7 of the primary transcript of the IKBKG gene (see FIG. 1). For example, the amino acid sequence: amino terminus-NQELRGNAAG-carboxy terminus [SEQ ID NO: 18], represents one embodiment of such an inventive IKBKGsv3 polypeptide wherein a first 5 amino acid region is encoded by a nucleotide sequence at the 3′ end of exon 2 of the IKBKG gene and a second 5 amino acid region is encoded by a nucleotide sequence directly after the novel splice junction. Preferably, at least 10 amino acids of the IKBKGsv3 polypeptide comprises a first continuous region of 2 to 8 amino acids that is encoded by nucleotides at the 3′ end of exon 2 and a second continuous region of 2 to 8 amino acids that is encoded by nucleotides at the 5′ end of exon 7.

In other embodiments, IKBKGsv1-specific antibodies are made using an IKBKGsv1 polypeptide that comprises at least 20, 30, 40 or 50 amino acids of the IKBKGsv1 sequence that corresponds to a junction polynucleotide region created by the alternative splicing of exon 4 to exon 6 of the primary transcript of the IKBKG gene. In each case the IKBKGsv1 polypeptides are selected to comprise a first continuous region of at least 5 to 15 amino acids that is encoded by nucleotides at the 3′ end of exon 4 and a second continuous region of 5 to 15 amino acids that is encoded by nucleotides directly after the novel splice junction.

In other embodiments, IKBKGsv2.1-specific antibodies are made using an IKBKGsv2.1 polypeptide that comprises at least 20, 30, 40 or 50 amino acids of the IKBKGsv2.1 sequence that corresponds to a junction polynucleotide region created by the alternative splicing of exon 3 to exon 6 of the primary transcript of the IKBKG gene. In each case the IKBKGsv2.1 polypeptides are selected to comprise a first continuous region of at least 5 to 15 amino acids that is encoded by nucleotides at the 3′ end of exon 3 and a second continuous region of 5 to 15 amino acids that is encoded by nucleotides directly after the novel splice junction.

In other embodiments, IKBKGsv2.2-specific antibodies are made using an IKBKGsv2.2 polypeptide that comprises at least 20, 30, 40, or 50 amino acids of the IKBKGsv2.2 sequence that corresponds to a polynucleotide region encoding amino acids, including and downstream of, the methionine codon located 3 nucleotides downstream of the 5′ end of exon 7 of the primary transcript of the IKBKG gene.

In other embodiments, IKBKGsv3-specific antibodies are made using an IKBKGsv3 polypeptide that comprises at least 20, 30, 40 or 50 amino acids of the IKBKGsv3 sequence that corresponds to a junction polynucleotide region created by the alternative splicing of exon 2 to exon 7 of the primary transcript of the IKBKG gene. In each case the IKBKGsv3 polypeptides are selected to comprise a first continuous region of at least 5 to 15 amino acids that is encoded by nucleotides at the 3′ end of exon 2 and a second continuous region of 5 to 15 amino acids that is encoded by nucleotides directly after the novel splice junction.

Antibodies to IKBKGsv1, IKBKGsv2.1, IKBKGsv2.2, or IKBKGsv3 have different uses, such as to identify the presence of IKBKGsv1, IKBKGsv2.1, IKBKGsv2.2, or IKBKGsv3, respectively, and to isolate IKBKGsv1, IKBKGsv2.1, IKBKGsv2.2, or IKBKGsv3 polypeptides, respectively. Identifying the presence of IKBKGsv1 can be used, for example, to identify cells producing IKBKGsv1. Such identification provides an additional source of IKBKGsv1 and can be used to distinguish cells known to produce IKBKGsv1 from cells that do not produce IKBKGsv1. For example, antibodies to IKBKGsv1 can distinguish human cells expressing IKBKGsv1 from human cells not expressing IKBKGsv1 or non-human cells (including bacteria) that do not express IKBKGsv1. Such IKBKGsv1 antibodies can also be used to determine the effectiveness of IKBKGsv1 ligands, using techniques well known in the art, to detect and quantify changes in the protein levels of IKBKGsv1 in cellular extracts, and in situ immunostaining of cells and tissues. In addition, the same above-described utilities also exist for IKBKGsv2.1-specific antibodies, IKBKGsv2.2-specific antibodies, and IKBKGsv3-specific antibodies.

Techniques for producing and using antibodies are well known in the art. Examples of such techniques are described in Ausubel, Current Protocols in Molecular Biology, John Wiley, 1987-1998; Harlow, et al., Antibodies, A Laboratory Manual, Cold Spring Harbor Laboratory, 1988; and Kohler, et al., 1975 Nature 256:495-7.

IKBKGsv1, IKBKGsv2.1, IKBKGsv2.2, and IKBKGsv3 Binding Assay

A number of compounds known to modulate NF-kappa-B activity have been disclosed (see for example, Baldwin, A. S., 1996, Annu. Rev. Immunol. 14, 649-681). However, only a limited number of compounds, such as PP2A and NBD peptides, have been implicated in the inhibition of IKBKG activity (Prajapati, S. and Gaynor, R., 2002, J. Bio. Chem. 277, 24331-24339; May, et. al., 2000, Science 289, 1550-1554). NBD peptides have been shown to have therapeutic effects in mouse models of acute inflammation (May, et al., 2000). Methods for screening compounds for their effects on IKBKG activity have also been disclosed (see for example, DiDonato, et. al., 1997, Nature 388, 548-554; May, et al., 2000; Burke, et. al., 2003, J. Biol. Chem. 278, 1450-1456). A person skilled in the art should be able to use these methods to screen IKBKGsv1, IKBKGsv2.1, IKBKGsv2.2, or IKBKGsv3 polypeptides for compounds that bind to, and in some cases functionally alter, each respective I-kappa-B-kinase-gamma isoform protein.

IKBKGsv1, IKBKGsv2.1, IKBKGsv2.2, IKBKGsv3, or fragments thereof, can be used in binding studies to identify compounds binding to or interacting with IKBKGsv1, IKBKGsv2.1, IKBKGsv2.2, IKBKGsv3, or fragments thereof, respectively. In one embodiment, the IKBKGsv1, or a fragment thereof, can be used in binding studies with IKBKG isoform protein, or a fragment thereof, to identify compounds that: bind to or interact with IKBKGsv1 and other IKBKG isoforms; bind to or interact with one or more other IKBKG isoforms and not with IKBKGsv1. A similar series of compound screens can, of course, also be performed using IKBKGsv2.1, IKBKGsv2.2, or IKBKGsv3 rather than, or in addition to, IKBKGsv1. Such binding studies can be performed using different formats including competitive and non-competitive formats. Further competition studies can be carried out using additional compounds determined to bind to IKBKGsv1, IKBKGsv2.1, IKBKGsv2.2, IKBKGsv3 or other IKBKG isoforms.

The particular IKBKGsv1, IKBKGsv2.1, IKBKGsv2.2, or IKBKGsv3 sequence involved in ligand binding can be identified using labeled compounds that bind to the protein and different protein fragments. Different strategies can be employed to select fragments to be tested to narrow down the binding region. Examples of such strategies include testing consecutive fragments about 15 amino acids in length starting at the N-terminus, and testing longer length fragments. If longer length fragments are tested, a fragment binding to a compound can be subdivided to further locate the binding region. Fragments used for binding studies can be generated using recombinant nucleic acid techniques.

In some embodiments, binding studies are performed using IKBKGsv1 expressed from a recombinant nucleic acid. Alternatively, recombinantly expressed IKBKGsv1 consists of the SEQ ID NO 5 amino acid sequence. In addition, binding studies are performed using IKBKGsv2.1 expressed from a recombinant nucleic acid. Alternatively, recombinantly expressed IKBKGsv2.1 consists of the SEQ ID NO 7 amino acid sequence. In addition, binding studies are performed using IKBKGsv2.2 expressed from a recombinant nucleic acid. Alternatively, recombinantly expressed IKBKGsv2.2 consists of the SEQ ID NO 9 amino acid sequence. In addition, binding studies are performed using IKBKGsv3 expressed from a recombinant nucleic acid. Alternatively, recombinantly expressed IKBKGsv3 consists of the SEQ ID NO 11 amino acid sequence.

Binding assays can be performed using individual compounds or preparations containing different numbers of compounds. A preparation containing different numbers of compounds having the ability to bind to IKBKGsv1, IKBKGsv2.1, IKBKGsv2.2, or IKBKGsv3 can be divided into smaller groups of compounds that can be tested to identify the compound(s) binding to IKBKGsv1, IKBKGsv2.1, IKBKGsv2.2, or IKBKGsv3, respectively.

Binding assays can be performed using recombinantly produced IKBKGsv1, IKBKGsv2.1, IKBKGsv2.2, or IKBKGsv3 present in different environments. Such environments include, for example, cell extracts and purified cell extracts containing a IKBKGsv1, IKBKGsv2.1, IKBKGsv2.2, or IKBKGsv3 recombinant nucleic acid; and also include, for example, the use of a purified IKBKGsv1, IKBKGsv2.1, IKBKGsv2.2, or IKBKGsv3 polypeptide produced by recombinant means which is introduced into different environments.

In one embodiment of the invention, a binding method is provided for screening for a compound able to bind selectively to IKBKGsv1. The method comprises the steps: providing a IKBKGsv1 polypeptide comprising SEQ ID NO 5; providing a IKBKG isoform polypeptide that is not IKBKGsv1; contacting the IKBKGsv1 polypeptide and the IKBKG isoform polypeptide that is not IKBKGsv1 with a test preparation comprising one or more test compounds; and then determining the binding of the test preparation to the IKBKGsv1 polypeptide and to the IKBKG isoform polypeptide that is not IKBKGsv1, wherein a test preparation that binds to the IKBKGsv1 polypeptide, but does not bind to IKBKG isoform polypeptide that is not IKBKGsv1, contains one or more compounds that selectively binds to IKBKGsv1.

In one embodiment of the invention, a binding method is provided for screening for a compound able to bind selectively to IKBKGsv2.1. The method comprises the steps: providing a IKBKGsv2.1 polypeptide comprising SEQ ID NO 7; providing a IKBKG isoform polypeptide that is not IKBKGsv2.1; contacting the IKBKGsv2.1 polypeptide and the IKBKG isoform polypeptide that is not IKBKGsv2.1 with a test preparation comprising one or more test compounds; and then determining the binding of the test preparation to the IKBKGsv2.1 polypeptide and to the IKBKG isoform polypeptide that is not IKBKGsv2.1, wherein a test preparation that binds to the IKBKGsv2.1 polypeptide, but does not bind to IKBKG isoform polypeptide that is not IKBKGsv2.1, contains one or more compounds that selectively binds to IKBKGsv2.1.

In another embodiment of the invention, a binding method is provided for screening for a compound able to bind selectively to IKBKGsv2.2. The method comprises the steps: providing a IKBKGsv2.2 polypeptide comprising SEQ ID NO 9; providing a IKBKG isoform polypeptide that is not IKBKGsv2.2; contacting the IKBKGsv2.2 polypeptide and the IKBKG isoform polypeptide that is not IKBKGsv2.2 with a test preparation comprising one or more test compounds; and then determining the binding of the test preparation to the IKBKGsv2.2 polypeptide and to the IKBKG isoform polypeptide that is not IKBKGsv2.2, wherein a test preparation that binds to the IKBKGsv2.2 polypeptide, but does not bind to IKBKG isoform polypeptide that is not IKBKGsv2.2, contains one or more compounds that selectively binds to IKBKGsv2.2.

In another embodiment of the invention, a binding method is provided for screening for a compound able to bind selectively to IKBKGsv3. The method comprises the steps: providing a IKBKGsv3 polypeptide comprising SEQ ID NO 11; providing a IKBKG isoform polypeptide that is not IKBKGsv3; contacting the IKBKGsv3 polypeptide and the IKBKG isoform polypeptide that is not IKBKGsv3 with a test preparation comprising one or more test compounds; and then determining the binding of the test preparation to the IKBKGsv3 polypeptide and to the IKBKG isoform polypeptide that is not IKBKGsv3, wherein a test preparation that binds to the IKBKGsv3 polypeptide, but does not bind to IKBKG isoform polypeptide that is not IKBKGsv3, contains one or more compounds that selectively binds to IKBKGsv3.

In another embodiment of the invention, a binding method is provided for screening for a compound able to bind selectively to a IKBKG isoform polypeptide that is not IKBKGsv1. The method comprises the steps: providing a IKBKGsv1 polypeptide comprising SEQ ID NO 5; providing a IKBKG isoform polypeptide that is not IKBKGsv1; contacting the IKBKGsv1 polypeptide and the IKBKG isoform polypeptide that is not IKBKGsv1 with a test preparation comprising one or more test compounds; and then determining the binding of the test preparation to the IKBKGsv1 polypeptide and the IKBKG isoform polypeptide that is not IKBKGsv1, wherein a test preparation that binds the IKBKG isoform polypeptide that is not IKBKGsv1, but does not bind the IKBKGsv1, contains a compound that selectively binds the IKBKG isoform polypeptide that is not IKBKGsv1. Alternatively, the above method can be used to identify compounds that bind selectively to a IKBKG isoform polypeptide that is not IKBKGsv2.1 by performing the method with IKBKGsv2.1 protein comprising SEQ ID NO 7. Alternatively, the above method can be used to identify compounds that bind selectively to a IKBKG isoform polypeptide that is not IKBKGsv2.2 by performing the method with IKBKGsv2.2 protein comprising SEQ ID NO 9. Alternatively, the above method can be used to identify compounds that bind selectively to a IKBKG isoform polypeptide that is not IKBKGsv3 by performing the method with IKBKGsv3 protein comprising SEQ ID NO 11.

The above-described selective binding assays can also be performed with a polypeptide fragment of IKBKGsv1, IKBKGsv2.1, or IKBKGsv3, wherein the polypeptide fragment comprises at least 10 consecutive amino acids that are coded by a nucleotide sequence that bridges the junction created by the splicing of the 3′ end of exon 4 to the 5′ end of exon 6 in the case of IKBKGsv1, by a nucleotide sequence that bridges the junction created by the splicing of the 3′ end of exon 3 to the 5′ end of exon 6 in the case of IKBKGsv2.1, or by the splicing of the 3′ end of exon 2 to the 5′ end of exon 7, in the case of IKBKGsv3. Similarly, the selective binding assays may also be performed using a polypeptide fragment of an IKBKG isoform polypeptide that is not IKBKGsv1, IKBKGsv2.1, or IKBKGsv3, wherein the polypeptide fragment comprises at least 10 consecutive amino acids that are coded by: a) a nucleotide sequence that is contained within exon 3, 4, 5, or 6, of the IKBKG gene; or b) a nucleotide sequence that bridges the junction created by the splicing of the 3′ end of exon 2 to the 5′ end of exon 3, the splicing of the 3′ end of exon 3 to the 5′ end of exon 4, the splicing of the 3′ end of exon 4 to the 5′ end of exon 5, the splicing of the 3′ end of exon 5 to the 5′ end of exon 6, or the splicing of the 3′ end of exon 6 to the 5′ end of exon 7 of the IKBKG gene.

IKBKG Functional Assays

IKBKG is an essential component of the I-kappa-B kinase complex that plays an integral role in the cascade leading to the activation of NF-kappa-B and the transcription of genes in response to harmful stimuli. The binding of IKBKG to the IKK complex activates the complex and phosphorylation of 1-kappa-B, leading to dissociation of 1-kappa-B from NF-kappa-B and the transport of NF-kappa-B to the nucleus. IKBKG activity also depends on its phosphorylation. The identification of IKBKGsv1, IKBKGsv2.1, IKBKGsv2.2, and IKBKGsv3 as splice variants of IKBKG provides a means for screening for compounds that bind to IKBKGsv1, IKBKGsv2.1, IKBKGsv2.2, and/or IKBKGsv3 protein thereby altering the ability of the IKBKGsv1, IKBKGsv2.1, IKBKGsv2.2, and/or IKBKGsv3 polypeptide to bind to the IKK complex, or to be phosphorylated. Assays involving a functional IKBKGsv1, IKBKGsv2.1, IKBKGsv2.2, or IKBKGsv3 polypeptide can be employed for different purposes, such as selecting for compounds active at IKBKGsv1, IKBKGsv2.1, IKBKGsv2.2, or IKBKGsv3; evaluating the ability of a compound to effect the phosphorylation of, or binding to the IKK complex, of each respective splice variant polypeptide; and mapping the activity of different IKBKGsv1, IKBKGsv2.1, IKBKGsv2.2, and IKBKGsv3 regions. IKBKGsv1, IKBKGsv2.1, IKBKGsv2.2, and IKBKGsv3 activity can be measured using different techniques such as: detecting a change in the intracellular conformation of IKBKGsv1, IKBKGsv2.1, IKBKGsv2.2, or IKBKGsv3; detecting a change in the intracellular location of IKBKGsv1, IKBKGsv2.1, IKBKGsv2.2, or IKBKGsv3; detecting the amount of binding of IKBKGsv1, IKBKGsv2.1, IKBKGsv2.2, or IKBKGsv3 to the IKK complex; or indirectly, by measuring the level of protein kinase activity of the IKK complex.

Recombinantly expressed IKBKGsv1, IKBKGsv2.1, IKBKGsv2.2, and IKBKGsv3 can be used to facilitate the determination of whether a compound is active at IKBKGsv1, IKBKGsv2.1, IKBKGsv2.2, and IKBKGsv3. For example, IKBKGsv1, IKBKGsv2.1, IKBKGsv2.2, and IKBKGsv3 can be expressed by an expression vector in a cell line and used in a co-culture growth assay, such as described in WO 99/59037, to identify compounds that bind to IKBKGsv1, IKBKGsv2.1, IKBKGsv2.2, and IKBKGsv3. For example, IKBKGsv1 can be expressed by an expression vector in a human kidney cell line 293 and used in a co-culture growth assay, such as described in U.S. Patent Application 20020061860, to identify compounds that bind to IKBKGsv1. A similar strategy can be used for IKBKGsv2.1, IKBKGsv2.2, or IKBKGsv3.

Techniques for measuring protein kinase activity are well known in the art (Huynh, et. al., 2000, J. Biol. Chem. 275, 25883-25891; Prajapati, S. and Gaynor, R. B., 2002, J. Biol. Chem. 277, 24331-24339). May et al. (2000, Science 289, 1550-1554) report methods for measuring the binding of IKBKG to the IKK complex. The method involves incubation of glutathione S-transferase tagged IKBKG with [³⁵S] methionine-labeled IKK catalytic subunits. A variety of other assays have been used to investigate the properties of IKBKG and therefore would also be applicable to the measurement of IKBKGsv1, IKBKGsv2.1, IKBKGsv2.2, or IKBKGsv3 functions.

IKBKGsv1, IKBKGsv2.1, IKBKGsv2.2, or IKBKGsv3 functional assays can be performed using cells expressing IKBKGsv1, IKBKGsv2.1, IKBKGsv2.2, or IKBKGsv3 at a high level. These proteins will be contacted with individual compounds or preparations containing different compounds. A preparation containing different compounds where one or more compounds affect IKBKGsv1, IKBKGsv2.1, IKBKGsv2.2, or IKBKGsv3 in cells over-producing IKBKGsv1, IKBKGsv2.1, IKBKGsv2.2, or IKBKGsv3 as compared to control cells containing expression vector lacking IKBKGsv1, IKBKGsv2.1, IKBKGsv2.2, or IKBKGsv3 coding sequences, can be divided into smaller groups of compounds to identify the compound(s) affecting IKBKGsv1, IKBKGsv2.1, IKBKGsv2.2, or IKBKGsv3 activity, respectively.

IKBKGsv1, IKBKGsv2.1, IKBKGsv2.2, or IKBKGsv3 functional assays can be performed using recombinantly produced IKBKGsv1, IKBKGsv2.1, IKBKGsv2.2, or IKBKGsv3 present in different environments. Such environments include, for example, cell extracts and purified cell extracts containing the IKBKGsv1, IKBKGsv2.1, IKBKGsv2.2, or IKBKGsv3 expressed from recombinant nucleic acid; and the use of a purified IKBKGsv1, IKBKGsv2.1, IKBKGsv2.2, or IKBKGsv3 produced by recombinant means that is introduced into a different environment suitable for measuring binding or kinase activity.

Modulating IKBKGsv1, IKBKGsv2.1, IKBKGsv2.2, and IKBKGsv3 Expression

IKBKGsv1, IKBKGsv2.1, IKBKGsv2.2, or IKBKGsv3 expression can be modulated as a means for increasing or decreasing IKBKGsv1, IKBKGsv2.1, IKBKGsv2.2, or IKBKGsv3 activity, respectively. Such modulation includes inhibiting the activity of nucleic acids encoding the IKBKG isoform target to reduce IKBKG isoform protein or polypeptide expressions, or supplying IKBKG nucleic acids to increase the level of expression of the IKBKG target polypeptide thereby increasing IKBKG activity.

Inhibition of IKBKGsv1 IKBKGsv2.1. IKBKGsv2.2 and IKBKGsv3 Activity

IKBKGsv1, IKBKGsv2.1, IKBKGsv2.2, or IKBKGsv3 nucleic acid activity can be inhibited using nucleic acids recognizing IKBKGsv1, IKBKGsv2.1, IKBKGsv2.2, or IKBKGsv3 nucleic acid and affecting the ability of such nucleic acid to be transcribed or translated. Inhibition of IKBKGsv1, IKBKGsv2.1, IKBKGsv2.2, or IKBKGsv3 nucleic acid activity can be used, for example, in target validation studies.

A preferred target for inhibiting IKBKGsv1, IKBKGsv2.1, IKBKGsv2.2, or IKBKGsv3 is mRNA stability and translation. The ability of IKBKGsv1, IKBKGsv2.1, IKBKGsv2.2, or IKBKGsv3 mRNA to be translated into a protein can be effected by compounds such as anti-sense nucleic acid, RNA interference (RNAi) and enzymatic nucleic acid.

Anti-sense nucleic acid can hybridize to a region of a target mRNA. Depending on the structure of the anti-sense nucleic acid, anti-sense activity can be brought about by different mechanisms such as blocking the initiation of translation, preventing processing of mRNA, hybrid arrest, and degradation of mRNA by RNAse H activity.

RNAi also can be used to prevent protein expression of a target transcript. This method is based on the interfering properties of double-stranded RNA derived from the coding regions of gene that disrupts the synthesis of protein from transcribed RNA.

Enzymatic nucleic acids can recognize and cleave other nucleic acid molecules. Preferred enzymatic nucleic acids are ribozymes.

General structures for anti-sense nucleic acids, RNAi and ribozymes, and methods of delivering such molecules, are well known in the art. Modified and unmodified nucleic acids can be used as anti-sense molecules, RNAi and ribozymes. Different types of modifications can effect certain anti-sense activities such as the ability to be cleaved by RNAse H, and can effect nucleic acid stability. Examples of references describing different anti-sense molecules, and ribozymes, and the use of such molecules, are provided in U.S. Pat. Nos. 5,849,902; 5,859,221; 5,852,188; and 5,616,459. Examples of organisms in which RNAi has been used to inhibit expression of a target gene include: C. elegans (Tabara, et al., 1999, Cell 99, 123-32; Fire, et al., 1998, Nature 391, 806-11), plants (Hamilton and Baulcombe, 1999, Science 286, 950-52), Drosophila (Hammond, et al., 2001, Science 293, 1146-50; Misquitta and Patterson, 1999, Proc. Nat. Acad. Sci. 96, 1451-56; Kennerdell and Carthew, 1998, Cell 95, 1017-26), and mammalian cells (Bernstein, et al., 2001, Nature 409, 363-6; Elbashir, et al., 2001, Nature 411, 494-8).

Increasing IKBKGsv1 IKBKGsv2.1 IKBKGsv2.2 and IKBKGsv3 Expression

Nucleic acids encoding for IKBKGsv1, IKBKGsv2.1, IKBKGsv2.2, or IKBKGsv3 can be used, for example, to cause an increase in IKBKG activity or to create a test system (e.g., a transgenic animal) for screening for compounds affecting IKBKGsv1, IKBKGsv2.1, IKBKGsv2.2, or IKBKGsv3 expression, respectively. Nucleic acids can be introduced and expressed in cells present in different environments.

Guidelines for pharmaceutical administration in general are provided in, for example, Remington's Pharmaceutical Sciences, 18^{th} Edition, supra, and Modern Pharmaceutics, 2^{nd} Edition, supra Nucleic acid can be introduced into cells present in different environments using in vitro, in vivo, or ex vivo techniques. Examples of techniques useful in gene therapy are illustrated in Gene Therapy & Molecular Biology: From Basic Mechanisms to Clinical Applications, Ed. Boulikas, Gene Therapy Press, 1998.

### EXAMPLES

Examples are provided below to further illustrate different features and advantages of the present invention. The examples also illustrate useful methodology for practicing the invention. These examples do not limit the claimed invention.

### Example 1

### Identification of IKBKGsv1, IKBKGsv2, and IKBKGsv3 Using Microarrays

To identify variants of the “normal” splicing of the exon regions encoding IKBKG, an exon junction microarray, comprising probes complementary to each splice junction resulting from splicing of the 10 exon coding sequences in IKBKG heteronuclear RNA (hnRNA), was hybridized to a mixture of labeled nucleic acid samples prepared from 44 different human tissue and cell line samples. Exon junction microarrays are described in PCT patent applications WO 02/18646 and WO 02/16650. Materials and methods for preparing hybridization samples from purified RNA, hybridizing a microarray, detecting hybridization signals, and data analysis are described in van't Veer, et al. (2002 Nature 415:530-536) and Hughes, et al. (2001 Nature Biotechnol. 19:342-7). Inspection of the exon junction microarray hybridization data (not shown) suggested that the structure of at least three of the exon junctions of IKBKG mRNA was altered in some of the tissues examined, suggesting the presence of IKBKG splice variant mRNA populations. Reverse transcription and polymerase chain reaction (RT-PCR) were then performed using oligonucleotide primers complementary to exons 2 and 7 to confirm the exon junction array results and to allow the sequence structure of the splice variants to be determined.

### Example 2

### Confirmation of IKBKGsv1, IKBKGsv2, and IKBKGsv3 Using RT-PCR

The structure of IKBKG mRNA in the region corresponding to exons 2 to 7 was determined for a panel of human tissue and cell line samples using an RT-PCR based assay. PolyA purified mRNA isolated from 44 different human tissue and cell line samples was obtained from BD Biosciences Clontech (Palo Alto, Calif.), Biochain Institute, Inc. (Hayward, Calif.), and Ambion Inc. (Austin, Tex.). RT-PCR primers were selected that were complementary to sequences in exon 2 and exon 7 of the reference exon coding sequences in IKBKG (NM_{—}003639). Based upon the nucleotide sequence of IKBKG mRNA, the IKBKG exon 2 and exon 7 primer set (hereafter IKBKG₂₋₇ primer set) was expected to amplify a 860 base pairs amplicon representing the “reference” IKBKG mRNA region. The IKBKG exon 2 forward primer has the sequence: 5′ TGTTGGATGAATAGGCACCTCTGGAAGA 3′ [SEQ ID NO: 19]; and the IKBKG exon 7 reverse primer has the sequence: 5′ TTCAGCTTATCGATCACCTCCTGTT TGG 3′ [SEQ ID NO: 20].

Twenty-five ng of polyA mRNA from each tissue was subjected to a one-step reverse transcription-PCR amplification protocol using the Qiagen, Inc. (Valencia, Calif.), One-Step RT-PCR kit, using the following conditions:

Cycling conditions were as follows:

```
  50° C. for 30 minutes;
  95° C. for 15 minutes;
35 cycles of:
  94° C. for 30 seconds;
63.5° C. for 40 seconds;
  72° C. for 50 seconds; then
  72° C. for 10 minutes.
```

RT-PCR amplification products (amplicons) were size fractionated on a 2% agarose gel. Selected amplicon fragments were manually extracted from the gel and purified with a Qiagen Gel Extraction Kit. Purified amplicon fragments were sequenced from each end (using the same primers used for RT-PCR) by Qiagen Genomics, Inc. (Bothell, Wash.).

At least three different RT-PCR amplicons were obtained from human mRNA samples using the IKBKG₂₋₇ primer set (data not shown). Every human tissue and cell line assayed exhibited the expected amplicon size of 860 base pairs for normally spliced IKBKG mRNA. However, in addition to the expected IKBKG amplicon of 860 base pairs, all cell lines assayed, except for ileocecum, also exhibited an amplicon of about 707 base pairs; fetal liver, brain, fetal brain, fetal lung, leukemia promyelocytic (HL-60), brain cerebellum, brain amygdala, brain caudate nucleus, brain thalmus, lymphoma Burkitts Raji, melanoma, lung carcinoma, brain cerebral cortex, epididymus, and brain hippocampus showed an additional amplicon of about 588 base pairs; and fetal brain, leukemia promyelocytic (HL-60), salivary gland, brain thalamus, lymphoma Burkitts Raji, melanoma, spinal cord, and epididymus also showed an amplicon of about 279 base pairs. These tissues in which IKBKGsv1, IKBKGsv2 and IKBKGsv3 mRNAs were detected are listed in Table 1.

TABLE 1

| Sample | IKBKGsv1 | IKBKGsv2 | IKBKGsv3 |
|---|---|---|---|
| Heart | x | | |
| Kidney | x | | |
| Liver | x | | |
| Brain | x | x | |
| Placenta | x | | |
| Lung | x | | |
| Fetal Brian | x | x | x |
| Leukemia Promyelocytic | x | x | x |
| (HL-60) | | | |
| Adrenal Gland | x | | |
| Fetal Liver | x | x | |
| Salivary Gland | x | | x |
| Pancreas | x | | |
| Skeletal Muscle | x | | |
| Brain Cerebellum | x | x | |
| Stomach | x | | |
| Trachea | x | | |
| Thyroid | x | | |
| Bone Marrow | x | | |
| Brain Amygdala | x | x | |
| Brain Caudate Nucleus | x | x | |
| Brain Corpus Callosum | x | | |
| Ileocecum | | | |
| Lymphoma Burkitt's (Raji) | x | x | x |
| Spinal Cord | x | | x |
| Lymph Node | x | | |
| Fetal Kidney | x | | |
| Uterus | x | | |
| Spleen | x | | |
| Brain Thalamus | x | x | x |
| Fetal Lung | x | x | |
| Testis | x | | |
| Melanoma (G361) | x | x | x |
| Lung Carcinoma (A549) | x | x | |
| Adrenal Medula, normal | x | | |
| Brain, Cerebral Cortex, | x | x | |
| normal; | | | |
| Descending Colon, normal | x | | |
| Prostate | x | | |
| Duodenum, normal | x | | |
| Epididymus, normal | x | x | x |
| Brain, Hippocamus, normal | x | x | |
| Ileum, normal | x | | |
| Interventricular Septum, | x | | |
| normal | | | |
| Jejunum, normal | x | | |
| Rectum, normal | x | | |

Sequence analysis of the about 707 base pair amplicon, corresponding to IKBKGsv1, revealed that this amplicon form results from the splicing of exon 4 of the IKBKG hnRNA to exon 6; that is, exon 5 coding sequence is completely absent. Sequence analysis of the about 588 base pair amplicon, corresponding to IKBKGsv2, revealed that this amplicon form results from the splicing of exon 3 of the IKBKG hnRNA to exon 6; that is, the exons 4 and 5 coding sequences are completely absent. Sequence analysis of the about 279 base pair amplicon, corresponding to IKBKGsv3, revealed that this amplicon form results from the splicing of exon 2 of the IKBKG hnRNA to exon 7; that is, the exons 3, 4, 5, and 6 coding sequences are completely absent. Thus, the RT-PCR results confirmed the junction probe microarray data reported in Example 1, which suggested that IKBKG mRNA is composed of a mixed population of molecules wherein in at least three of the IKBKG mRNA splice junctions are altered.

### Example 3

### Cloning of IKBKGsv1, IKBKGsv2.1, IKBKGsv2.2, and IKBKGsv3

Microarray and RT-PCR data indicate that in addition to the normal IKBKG reference mRNA sequence, NM_{—}003639, encoding IKBKG protein, NP_{—}003630, three novel splice variant forms of IKBKG mRNA also exist in many tissues.

Clones having nucleotide sequence comprising the splice variants identified in Example 2 (hereafter referred to as IKBKGsv1, IKBKGsv2.1, IKBKGsv2.2, or IKBKGsv3) are isolated using a 5′ “forward” IKBKG primer and a 3′ “reverse” IKBKG primer, to amplify and clone the entire IKBKGsv1, IKBKGsv2.1, IKBKGsv2.2, or IKBKGsv3 mRNA coding sequences, respectively. The same 5′ “forward” primer is designed for isolation of full length clones corresponding to the IKBKGsv1, IKBKGsv2.1, and IKBKGsv3 splice variants and has the nucleotide sequence of 5′ ATGAATAGGCAC CTCTGGAAGAGCCAAC 3′ [SEQ ID NO 21]. The 5′ “forward” IKBKGsv2.2 primer is designed to have the nucleotide sequence of 5′ ATGCAGCTGGAAGATCTCAAA CAGCAG 3′ [SEQ ID NO 22]. The same 3′ “reverse” primer is designed for isolation of full length clones corresponding to the IKBKGsv1 and IKBKGsv2.2 splice variants and has the nucleotide sequence of 5′ CTACTCAATGCACTCCATGACATGTAT 3′ [SEQ ID NO 23]. The 3′ “reverse” IKBKGsv2.1 primer is designed to have the nucleotide sequence of 5′ TCACTGCCCACCACGCTGCTCTTGATG 3′ [SEQ ID NO 24]. The 3′ “reverse” IKBKGsv3 primer is designed to have the nucleotide sequence of 5′ TTATCGATCACCT CCTGTTTGGCCACC 3′ [SEQ ID NO 25].

RT-PCR

The IKBKGsv1, IKBKGsv2.1, IKBKGsv2.2, and IKBKGsv3 cDNA sequences are cloned using a combination of reverse transcription (RT) and polymerase chain reaction (PCR). More specifically, about 25 ng of fetal brain polyA mRNA (BD Biosciences Clontech, Palo alto, CA) is reverse transcribed using Superscript II (Gibco/Invitrogen, Carlsbad, Calif.) and oligo d(T) primer (RESGEN/Invitrogen, Huntsville, Ala.) according to the Superscript II manufacturer's instructions. For PCR, 1 μl of the completed RT reaction is added to 40 μl of water, 5 μl of 10× buffer, 1 μl of dNTPs and 1 μl of enzyme from the Clontech (Palo Alto, Calif.) Advantage 2 PCR kit. PCR is done in a Gene Amp PCR System 9700 (Applied Biosystems, Foster City, Calif.) using the IKBKG “forward” and “reverse” primers. After an initial 94° C. denaturation of 1 minute, 35 cycles of amplification are performed using a 30 second denaturation at 94° C. followed by a 40 second annealing at 63.5° C. and a 50 second synthesis at 72° C. The 35 cycles of PCR are followed by a 10 minute extension at 72° C. The 50 μl reaction is then chilled to 4° C. 10 μl of the resulting reaction product is run on a 1% agarose (Invitrogen, Ultra pure) gel stained with 0.3 μg/ml ethidium bromide (Fisher Biotech, Fair Lawn, N.J.). Nucleic acid bands in the gel are visualized and photographed on a UV light box to determined if the PCR has yielded products of the expected size, in the case of the predicted IKBKGsv1, IKBKGsv2.1, IKBKGsv2.2, and IKBKGsv3 mRNAs, products of about 1107, 486, 489, and 267 bases, respectively. The remainder of the 50 μl PCR reactions from fetal brain is purified using the QIAquik Gel extraction Kit (Qiagen, Valencia, Calif.) following the QIAquik PCR Purification Protocol provided with the kit. An about 50 μl of product obtained from the purification protocol is concentrated to about 6 μl by drying in a Speed Vac Plus (SC 110A, from Savant, Holbrook, N.Y.) attached to a Universal Vacuum Sytem 400 (also from Savant) for about 30 minutes on medium heat.

Cloning of RT-PCR Products

About 4 Tl of the 6 Tl of purified IKBKGsv1, IKBKGsv2.1, IKBKGsv2.2, and IKBKGsv3 RT-PCR products from fetal brain are used in a cloning reaction using the reagents and instructions provided with the TOPO TA cloning kit (Invitrogen, Carlsbad, Calif.). About 2 Tl of the cloning reaction is used following the manufacturer's instructions to transform TOP 10 chemically competent E. coli provided with the cloning kit. After the 1 hour recovery of the cells in SOC medium (provided with the TOPO TA cloning kit), 200 Tl of the mixture is plated on LB medium plates (Sambrook, et al., in Molecular Cloning, A Laboratory Manual, 2^{nd} Edition, Cold Spring Harbor Laboratory Press, 1989) containing 100 Tg/ml Ampicillin (Sigma, St. Louis, Mo.) and 80 Tg/ml X-GAL (5-Bromo-4-chloro-3-indoyl B-D-galactoside, Sigma, St. Louis, Mo.). Plates are incubated overnight at 37° C. White colonies are picked from the plates into 2 ml of 2×LB medium. These liquid cultures are incubated overnight on a roller at 37° C. Plasmid DNA is extracted from these cultures using the Qiagen (Valencia, Calif.) Qiaquik Spin Miniprep kit. Twelve putative IKBKGsv1, IKBKGsv2.1, IKBKGsv2.2, and IKBKGsv3 clones, respectively are identified and prepared for a PCR reaction to confirm the presence of the expected IKBKGsv1 exon 4 to exon 6, IKBKGsv2.1 exon 3 to exon 6, IKBKGsv2.2 exon 7 to exon 10, and IKBKGsv3 exon 2 to exon 7 splice variant structures. A 25 Tl PCR reaction is performed as described above (RT-PCR section) to detect the presence of IKBKGsv1, except that the reaction includes miniprep DNA from the TOPO TA/IKBKGsv1 ligation as a template. An additional 25 Tl PCR reaction is performed as described above (RT-PCR section) to detect the presence of IKBKGsv2.1, except that the reaction includes miniprep DNA from the TOPO TA/IKBKGsv2.1 ligation as a template. An additional 25 Tl PCR reaction is performed as described above (RT-PCR section) to detect the presence of IKBKGsv2.2, except that the reaction includes miniprep DNA from the TOPO TA/IKBKGsv2.2 ligation as a template. An additional 25 Tl PCR reaction is performed as described above (RT-PCR section) to detect the presence of IKBKGsv3, except that the reaction includes miniprep DNA from the TOPO TA/IKBKGsv3 ligation as a template. About 10 Tl of each 25 Tl PCR reaction is run on a 1% Agarose gel and the DNA bands generated by the PCR reaction are visualized and photographed on a UV light box to determine which minipreps samples have PCR product of the size predicted for the corresponding IKBKGsv1, IKBKGsv2.1, IKBKGsv2.2, and IKBKGsv3 splice variant mRNAs. Clones having the IKBKGsv1 structure are identified based upon amplification of an amplicon band of 1107 basepairs, whereas a normal reference IKBKG clone will give rise to an amplicon band of 1260 basepairs. Clones having the IKBKGsv2.1 structure are identified based upon amplification of an amplicon band of 486 basepairs, whereas a normal reference IKBKG clone would give rise to an amplicon band of 758 basepairs. Clones having the IKBKGsv2.2 structure are identified based upon amplification of an amplicon band of 489 basepairs. Clones having the IKBKGsv3 structure are identified based upon amplification of an amplicon band of 267 basepairs, whereas a normal reference IKBKG clone would give rise to an amplicon band of 848 basepairs. DNA sequence analysis of the IKBKGsv1, IKBKGsv2.1, IKBKGsv2.2, or IKBKGsv3 cloned DNAs confirm a polynucleotide sequence representing the deletion of exon 5 in the case of IKBKGsv1; the deletion of exons 4 and 5 in the case of IKBKGsv2.1; the absence of exons 1, 2, 3, 4, 5 and 6, and the first 3 nucleotides of exon 7 in the case of IKBKGsv2.2, or the deletion of exon 3, 4, 5, and 6 in the case of IKBKGsv3.

The polynucleotide sequence of IKBKGsv1 mRNA (SEQ ID NO 4) contains an open reading frame that encodes a IKBKGsv1 protein (SEQ ID NO 5) similar to the reference IKBKG protein (NP_{—}003630), but lacking the amino acids encoded by a 153 base pair region corresponding to exon 5 of the full length coding sequence of reference IKBKG mRNA (NM_{—}003639). The deletion of the 153 base pair region results in a protein translation reading frame that is in alignment in comparison to the reference IKBKG protein reading frame. Therefore the IKBKGsv1 protein is only missing an internal 51 amino acid region as compared to the reference IKBKG (NP_{—}003630).

The polynucleotide sequence of IKBKGsv2 mRNA contains two open reading frames that encode an amino terminal and a carboxy terminal protein, referred to herein as IKBKGsv2.1 and IKBKGsv2.2, respectively. SEQ ID NO 6 encodes the amino terminal IKBKGsv2.1 protein (SEQ ID NO 7), similar to the reference IKBKG protein (NP_{—}003630), but lacking the amino acids encoded by a 272 base pair region corresponding to exons 4 and 5 of the full length coding sequence of reference IKBKG mRNA (NM_{—}003639). The alternative spliced IKBKGsv2.1 mRNA not only deletes a 272 base pair region corresponding to exons 4 and 5, but also results in a protein reading frame shift at the exon 3/exon 6 splice junction, creating a protein translation reading frame that is out of alignment in comparison to the reference IKBKG protein reading frame. This shift in reading frame creates a premature termination codon, resulting in the production of an altered and shorter IKBKGsv2.1 protein as compared to the reference IKBKG protein (NP_{—}003630). IKBKGsv2.2 polynucleotide (SEQ ID NO 8) encodes the carboxy terminal IKBKGsv2.2 protein (SEQ ID NO 9), similar to the reference IKBKG protein (NP_{—}003630), but lacking the first 257 amino acids of the reference IKBKG protein (NP_{—}003630) due to utilization of a translation initiation AUG codon downstream from the AUG initiation codon utilized by the reference IKBKG protein (NP_{—}003630).

The polynucleotide sequence of IKBKGsv3 mRNA (SEQ ID NO 10) contains an open reading frame that encodes a IKBKGsv3 protein (SEQ ID NO 11) similar to the reference IKBKG protein (NP_{—}003630), but lacking amino acids encoded by exons 3, 4, 5, and 6 of the full length coding sequence of reference IKBKG mRNA (NM_{—}003639). The alternative splicing of exon 2 to exon 7 not only deletes a 581 base pair region corresponding to exons 3-6, but also results in a protein reading frame shift at the novel exon 2/exon 7 splice junction, creating a protein translation reading frame that is out of alignment in comparison to the reference IKBKG protein reading frame. This shift in reading frame creates a premature termination codon, resulting in the production of an altered and shorter IKBKGsv3 protein as compared to the reference IKBKG protein (NP_{—}003630).

All patents, patent publications, and other published references mentioned herein are hereby incorporated by reference in their entireties as if each had been individually and specifically incorporated by reference herein. While preferred illustrative embodiments of the present invention are shown and described, one skilled in the art will appreciate that the present invention can be practiced by other than the described embodiments, which are presented for purposes of illustration only and not by way of limitation. Various modifications may be made to the embodiments described herein without departing from the spirit and scope of the present invention. The present invention is limited only by the claims that follow.

What is claimed:

1. A purified human nucleic acid comprising SEQ ID NO 4, or the complement thereof.

2. The purified nucleic acid of claim 1, wherein said nucleic acid comprises a region encoding SEQ ID NO 5.

3. The purified nucleic acid of claim 1, wherein said nucleotide sequence encodes a polypeptide consisting of SEQ ID NO 5.

4. A purified polypeptide comprising SEQ ID NO 5.

5. The polypeptide of claim 4, wherein said polypeptide consists of SEQ ID NO 5.

6. An expression vector comprising a nucleotide sequence encoding SEQ ID NO 5, wherein said nucleotide sequence is transcriptionally coupled to an exogenous promoter.

7. The expression vector of claim 6, wherein said nucleotide sequence encodes a polypeptide consisting of SEQ ID NO 5.

8. The expression vector of claim 6, wherein said nucleotide sequence comprises SEQ ID NO 4.

9. The expression vector of claim 6, wherein said nucleotide sequence consists of SEQ ID NO 4.

10. A method for screening for a compound able to bind to IKBKGsv1 comprising the steps of:
(a) expressing a polypeptide comprising SEQ ID NO 5 from recombinant nucleic acid;
(b) providing to said polypeptide a test preparation comprising one or more test compounds; and
(c) measuring the ability of said test preparation to bind to said polypeptide.

11. The method of claim 10, wherein said steps (b) and (c) are performed in vitro.

12. The method of claim 10, wherein said steps (a), (b), and (c) are performed using a whole cell.

13. The method of claim 10, wherein said polypeptide is expressed from an expression vector.

14. The method of claim 10, wherein said polypeptide consists of SEQ ID NO 5.

15. A method of screening for compounds able to bind selectively to IKBKGsv1 comprising the steps of:
(a) providing a IKBKGsv1 polypeptide comprising SEQ ID NO 5;
(b) providing one or more IKBKG isoform polypeptides that are not IKBKGsv1;
(c) contacting said IKBKGsv1 polypeptide and said IKBKG isoform polypeptide that is not IKBKGsv1 with a test preparation comprising one or more compounds; and
(d) determining the binding of said test preparation to said IKBKGsv1 polypeptide and to said IKBKG isoform polypeptide that is not IKBKGsv1, wherein a test preparation that binds to said IKBKGsv1 polypeptide, but does not bind to said IKBKG polypeptide that is not IKBKGsv1, contains a compound that selectively binds said IKBKGsv1 polypeptide.

16. The method of claim 15, wherein said IKBKGsv1 polypeptide is obtained by expression of said polypeptide from an expression vector comprising a polynucleotide encoding SEQ ID NO 5.

17. The method of claim 16, wherein said polypeptide consists of SEQ ID NO 5.

18. A method for screening for a compound able to bind to or interact with a IKBKGsv1 protein or a fragment thereof comprising the steps of:
(a) expressing a IKBKGsv1 polypeptide comprising SEQ ID NO 5 or fragment thereof from a recombinant nucleic acid;
(b) providing to said polypeptide a labeled IKBKG ligand that binds to said polypeptide and a test preparation comprising one or more compounds; and
(c) measuring the effect of said test preparation on binding of said labeled IKBKG ligand to said polypeptide, wherein a test preparation that alters the binding of said labeled IKBKG ligand to said polypeptide contains a compound that binds to or interacts with said polypeptide.

19. The method of claim 18, wherein said steps (b) and (c) are performed in vitro.

20. The method of claim 18, wherein said steps (a), (b) and (c) are performed using a whole cell

21. The method of claim 18, wherein said polypeptide is expressed from an expression vector

22. The method of claim 18, wherein said IKBKGsv1 ligand is an IKBKG inhibitor.

23. The method of claim 21, wherein said expression vector comprises SEQ ID NO 4 or a fragment of SEQ ID NO 4.

24. The method of claim 21, wherein said polypeptide comprises SEQ ID NO 4 or a fragment of SEQ ID NO 4.
